(19) United States
(12) Patent Application Publication
(10) Pub. No.: US 2004/0167229 A1
(43) Pub. Date: Aug. 26, 2004

(54) Method of lowering CRP and reducing systemic inflammation

(76) Inventors: Rebecca Bakker-Arkema, Ann Arbor, MI (US); Ok Hwang, Ann Arbor, MI (US); Sotirios Karathanasis, Carmel, IN (US); Diane MacDougall, Ann Arbor, MI (US)

Correspondence Address: Cynthia M. Bott, Warner-Lambert Company, 2800 Plymouth Road, Ann Arbor, MI, 48105, US

(21) Appl. No.: 10/712,859

(22) Filed: Nov. 13, 2003

Related U.S. Application Data
(60) Provisional application No. 60/426,565, filed on Nov. 15, 2002.

(51) Int. Cl. A61K 31/12; A61K 31/10; A01N 31/14

(52) U.S. Cl. 514/675; 514/712; 514/722

(57) ABSTRACT

Disclosed are methods of lowering plasma CRP levels, reducing systemic inflammation, and inhibiting proinflammatory cytokine induced CRP production by administering an effective amount of a substituted dialkyl ether, substituted alkyl, substituted aryl-alkyl, substituted dialkyl thioether, substituted dialkyl ketone, substituted-alkyl, or a pharmaceutically acceptable salt of the substituted dialkyl ether, substituted alkyl, substituted aryl-alkyl, substituted dialkyl thioether, substituted dialkyl ketone, or substituted-alkyl or a pharmaceutical composition comprising a substituted dialkyl ether, substituted alkyl, substituted aryl-alkyl, substituted dialkyl thioether, substituted dialkyl ketone, substituted-alkyl.

### PRIORITY INFORMATION

This application claims benefit of U.S. provisional application Ser. No. 60/426,565, filed Nov. 15, 2002, herein incorporated by reference in its entirety.

### FIELD OF THE INVENTION

This invention relates to methods of lowering C-reactive protein (CRP), of reducing systemic inflammation and of inhibiting proinflammatory cytokine induced CRP production, comprising administering an effective amount of a substituted dialkyl ether, substituted alkyl, substituted aryl-alkyl, substituted dialkyl thioether, substituted dialkyl ketone, substituted-alkyl, or a pharmaceutically acceptable salt of the substituted dialkyl ether, substituted alkyl, substituted aryl-alkyl, substituted dialkyl thioether, substituted dialkyl ketone, or substituted-alkyl, or a pharmaceutical composition of the substituted dialkyl ether, substituted alkyl, substituted aryl-alkyl, substituted dialkyl thioether, substituted dialkyl ketone, substituted-alkyl, or a pharmaceutical composition of the pharmaceutically acceptable salt of the substituted dialkyl ether, substituted alkyl, substituted aryl-alkyl, substituted dialkyl thioether, substituted dialkyl ketone, or substituted-alkyl.

### BACKGROUND OF THE INVENTION

Vascular diseases such as coronary heart disease, stroke, restenosis, and peripheral vascular disease, remain the leading cause of death and disability throughout the world. Over 700,000 people die each year in the US alone from diseases of the heart and an additional 166,000 die of cerebrovascular diseases, especially stroke. Many of the deaths occur from the approximately 1.5 million people who annually suffer a myocardial infarction and/or develop congestive heart failure. Myocardial infarction is most commonly precipitated by rupture of an atherosclerotic plaque in the coronary arteries that in turn leads to a clot (thrombus) forming at the site of the rupture. Myocardial infarction results when the clot occludes the vessel (i.e., coronary artery) and blood flow to the myocardium is sufficiently impaired or interrupted for long enough to result in tissue death. Preventing plaque rupture and/or lessening the likelihood of plaque rupture is the scientific rationale for treating common coronary risk factors such as elevated serum cholesterol, smoking, hypertension, and diabetes mellitus. The inflammatory process has been strongly implicated in the initiation and progression of atherosclerosis. Basic and epidemiological research suggests that inflammation within the atherosclerotic plaque affects its stability. Hence, identifying a method that reduces inflammation could be an important means of preventive treatment for coronary and other vascular diseases including stroke, peripheral artery disease, and other types of vascular insufficiency such as transient ischemic attacks (TIAs); vertebro-basilar insufficiency; claudication; gangrene of extremities; Raynaud's disease; impotence related to aorto-iliac disease; mesenteric insufficiency; and other forms of abdominal angina and angina pectoris.

CRP is a marker of systemic inflammation (i.e., levels of CRP correlate with the level of systemic inflammation in an individual). CRP is produced mainly in the liver in response to proinflammatory cytokines, as part of an acute phase response. Increased levels of CRP have been independently associated with increased risk of coronary heart disease. In addition to its association with increased risk of coronary heart disease, elevated levels of CRP are found in other populations including, but not limited to persons who smoke, have metabolic syndrome, type II diabetes melitis, glucose intolerance, osteoarthritis or systemic inflammatory disease such as rheumatoid arthritis, soriatic arthritis, spondyloarthropathy or vasculitis.

Some drugs, including lipid-altering drugs, have been shown to reduce CRP. For example several studies of the effect of statins on CRP have shown significant reductions in CRP that are not correlated with changes in LDL-C. While the general consensus is that statins lower CRP, some studies have shown that statins have only a modest or no effect on CRP levels. In addition, studies of the effects of lipid lowering fibrates on CRP have been inconclusive, with some studies showing significant reductions and others failing to find a reduced CRP level. Thus, it is not possible, at this time, to predict in advance which lipid-lowering drugs will also lower CRP levels. Because lowering of CRP is associated with decreased risk of adverse coronary events, additional drugs that lower CRP are needed. In addition, because systemic inflammation is a component of many diseases, additional drugs that reduce systemic inflammation are needed.

We have previously found that certain carboxyalkyethers are effective in lowering plasma concentrations of Lp(a), triglycerides and elevating HDL-cholesterol, these compounds are described in U.S. Pat. No. 5,648,387, incorporated by reference herein in its entirety.

### SUMMARY OF THE INVENTION

Generally the present invention comprises a method for lowering plasma CRP levels comprising administering to a mammal, in need thereof, an effective amount of a substituted dialkyl ether, substituted alkyl, substituted aryl-alkyl, substituted dialkyl thioether, substituted dialkyl ketone, substituted-alkyl, or a pharmaceutically acceptable salt of the substituted dialkyl ether, substituted alkyl, substituted aryl-alkyl, substituted dialkyl thioether, substituted dialkyl ketone, or substituted-alkyl.

One embodiment of the invention is a method for lowering plasma CRP levels comprising administering to a mammal, in need thereof, an effective amount of a compound of the Formula I:

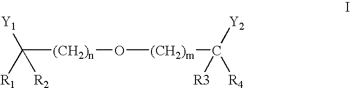

wherein n and m independently are integers from 2 to 9; R₁, R₂, R₃, and R₄ independently are C₁-C₆ alkyl, C₂-C₆ alkenyl, C₂-C₆ alkynyl, and R₁ and R₂ together with the carbon to which they are attached, and R₃ and R₄ together with the carbon to which they are attached, independently can complete a carbocyclic ring having from 3 to 6 carbons; Y₁ and Y₂ independently are COOH, CHO, tetrazole, and COOR₅ where R₅ is C₁-C₆ alkyl, C₂-C₆ alkenyl, C₂-C₆ alkynyl, and where the alkyl, alkenyl, and alkynyl groups may be substituted with one or two groups selected from halo, hydroxy, C₁-C₆ alkoxy, and phenyl, or a pharmaceutically acceptable salt thereof.

Another embodiment of the invention is a method for lowering plasma CRP levels comprising administering to a mammal, in need thereof, an effective amount of 6,6′-oxybis(2,2-dimethylhexanoic acid).

Another embodiment of the invention is a method for lowering plasma CRP levels comprising administering to a mammal, in need thereof, an effective amount of a pharmaceutical composition comprising a substituted dialkyl ether, substituted alkyl, substituted aryl-alkyl, substituted dialkyl thioether, substituted dialkyl ketone, substituted-alkyl, or a pharmaceutically acceptable salt of the substituted dialkyl ether, substituted alkyl, substituted aryl-alkyl, substituted dialkyl thioether, substituted dialkyl ketone, and a pharmaceutically acceptable diluent, carrier, or excipient.

An embodiment of the invention is a method for lowering plasma CRP levels comprising administering to a mammal, in need thereof, an effective amount of a pharmaceutical composition comprising a compound of formula 1 or a pharmaceutically acceptable salt thereof and a pharmaceutically acceptable diluent, carrier, or excipient.

Another embodiment of the invention is a method for lowering plasma CRP levels comprising administering to a mammal, in need thereof, an effective amount of a pharmaceutical composition comprising 6,6′-oxybis(2,2-dimethylhexanoic acid).

More specific embodiments of the invention include the methods of lowering plasma CRP levels described above wherein the compound inhibits proinflammatory cytokine induced CRP production.

Additional embodiments of the invention include methods of lowering plasma CRP levels described above wherein the mammal is a human.

Another embodiment of the invention is a method for reducing systemic inflammation in a mammal comprising administering to the mammal an effective amount of a substituted dialkyl ether, substituted alkyl, substituted aryl-alkyl, substituted dialkyl thioether, substituted dialkyl ketone, substituted-alkyl, or a pharmaceutically acceptable salt of the substituted dialkyl ether, substituted alkyl, substituted aryl-alkyl, substituted dialkyl thioether, substituted dialkyl ketone, or substituted-alkyl.

Another embodiment of the invention is a method for reducing systemic inflammation in a mammal comprising administering to the mammal an effective amount of a compound of formula 1 or a pharmaceutically acceptable salt thereof.

Another embodiment of the invention is a method for reducing systemic inflammation in a mammal comprising administering to the mammal an effective amount of 6,6′-oxybis(2,2-dimethylhexanoic acid).

Another embodiment of the invention is a method for reducing systemic inflammation in a mammal comprising administering to the mammal an effective amount of a pharmaceutical composition comprising a substituted dialkyl ether, substituted alkyl, substituted aryl-alkyl, substituted dialkyl thioether, substituted dialkyl ketone, substituted-alkyl, or a pharmaceutically acceptable salt of the substituted dialkyl ether, substituted alkyl, substituted aryl-alkyl, substituted dialkyl thioether, substituted dialkyl ketone, or substituted-alkyl, and a pharmaceutically acceptable diluent, carrier, or excipient.

Another embodiment of the invention is a method for reducing systemic inflammation in a mammal comprising administering to the mammal an effective amount of a pharmaceutical composition comprising a compound of formula 1 or a pharmaceutically acceptable salt thereof and a pharmaceutically acceptable diluent, carrier, or excipient.

Another embodiment of the invention is a method for reducing systemic inflammation in a mammal comprising administering to the mammal an effective amount of a pharmaceutical composition comprising 6,6′-oxybis(2,2-dimethylhexanoic acid).

More specific embodiments of the invention include the methods of for reducing systemic inflammation described above wherein the compound inhibits proinflammatory cytokine induced CRP production.

Additional embodiments of the invention include methods for reducing systemic inflammation described above wherein the mammal is a human.

Another embodiment of the invention is a method for inhibiting proinflammatory cytokine induced CRP production in a mammal comprising administering to the mammal an effective amount of a substituted dialkyl ether, substituted alkyl, substituted aryl-alkyl, substituted dialkyl thioether, substituted dialkyl ketone, substituted-alkyl, or a pharmaceutically acceptable salt of the substituted dialkyl ether, substituted alkyl, substituted aryl-alkyl, substituted dialkyl thioether, substituted dialkyl ketone, or substituted-alkyl.

Another embodiment of the invention is a method for inhibiting proinflammatory cytokine induced CRP production in a mammal comprising administering to the mammal an effective amount of a compound of formula 1 or a pharmaceutically acceptable salt thereof.

Another embodiment of the invention is a method for inhibiting proinflammatory cytokine induced CRP production in a mammal comprising administering to the mammal an effective amount of 6,6′-oxybis(2,2-dimethylhexanoic acid).

Another embodiment of the invention is a method for inhibiting proinflammatory cytokine induced CRP production in a mammal comprising administering to the mammal an effective amount of a pharmaceutical composition comprising a substituted dialkyl ether, substituted alkyl, substituted aryl-alkyl, substituted dialkyl thioether, substituted dialkyl ketone, substituted-alkyl, or a pharmaceutically acceptable salt of the substituted dialkyl ether, substituted alkyl, substituted aryl-alkyl, substituted dialkyl thioether, substituted dialkyl ketone, or substituted-alkyl, and a pharmaceutically acceptable diluent, carrier, or excipient.

Another embodiment of the invention is a method for inhibiting proinflammatory cytokine induced CRP production in a mammal comprising administering to the mammal an effective amount of a pharmaceutical composition comprising a compound of formula 1 or a pharmaceutically acceptable salt thereof and a pharmaceutically acceptable diluent, carrier, or excipient.

Another embodiment of the invention is a method for inhibiting proinflammatory cytokine induced CRP production in a mammal comprising administering to the mammal an effective amount of a pharmaceutical composition comprising 6,6′-oxybis(2,2-dimethylhexanoic acid).

Additional embodiments of the invention include methods for inhibiting proinflammatory cytokine induced CRP production described above wherein the mammal is a human.

One or more of the substituted dialkyl ether, substituted alkyl, substituted aryl-alkyl, substituted dialkyl thioether, substituted dialkyl ketone, substituted-alkyl, or a pharmaceutically acceptable salt of the substituted dialkyl ether, substituted alkyl, substituted aryl-alkyl, substituted dialkyl thioether, substituted dialkyl ketone, or substituted-alkyl described herein can be used in the preparation of a medicament for lowering plasma CRP levels in a mammal, for reducing systemic inflammation in a mammal, or for inhibiting proinflammatory cytokine induced CRP production in a mammal.

### BREIF DESCRIPTION OF THE FIGURES

FIG. 1 (FIG. 1). PLC/PRF/5 Human Hepatoma Cells were treated, as described in Example 16, with or without 6,6′-oxybis(2,2-dimethylhexanoic acid) at the doses indicated and the CRP levels in the cell media were determined. Bars represent the mean±SEM of each treatment group.

FIG. 2 (FIG. 2). Patients were treated, as described in Example 17, with the doses indicated on the X-axis (mg/day). Median CRP Percent Change, calculated as described in Example 17, is indicated on the Y-axis.

### DETAILED DESCRIPTION OF THE INVENTION

Abbreviations

The following list contains abbreviations used within the schemes and text:

| | |
|---|---|
| ANCOVA | Analysis of Covariance |
| cc | cubic centimeter |
| CRP | C-reactive protein |
| COOEt | Ethoxycarbonyl |
| Et | Ethyl |
| EP | European pharmacopea |
| HDL | high-density lipoprotein |
| HDL-C | high-density lipoprotein-cholesterol |
| i-Bu | isobutyl |
| i-Pr | isopropyl |
| IL-6 | Interleukin-6 |
| LDL | low-density lipoprotein |
| LDL-C | low-density lipoprotein-cholesterol |
| mp | melting point |
| NCEP | National Cholesterol Education Program |
| NF | National Formulary |
| n-Bu | Normal-butyl |
| n-hexyl | normal-hexyl |
| n-Pr | normal-propyl |
| qs | quantity sufficient |
| TG | Triglyceride |

Definitions and Usage of Terms

“Alkyl” means a substituted or unsubstituted, straight or branched hydrocarbon radical and includes for example methyl, ethyl, n-propyl, isopropyl, n-butyl, tert-butyl, sec-butyl, isobutyl, tert-butyl, n-hexyl, and 2-methylpentyl. Typical substituted alkyl groups are chlormethyl, 3-hydroxyhexyl, 4-phenylbutyl, 2-iodopentyl, isopropoxymethyl, and the like.

“Alkoxy” means an alkyl or alkenyl linked through oxygen (i.e., —O-alkyl or —O-alkenyl), including for example, methoxy, ethoxy, propoxy, isopropoxy and allyloxy.

“Alkenyl” is an unsubstituted or substituted, straight or branched hydrocarbon chain radical with one or more carbon-carbon double bonds, including, for example, vinyl, allyl, butenyl, 3-chloro-4-hexenyl, and 2-phenyl-3-pentenyl

“Alkynyl” is an unsubstituted or substituted hydrocarbon chain radical with at least one carbon-carbon triple bond. Typical groups include, for example, ethynyl, 2-methoxyethynyl, 2-bromoethynyl, 6-phenyl-3-hexynyl.

“Halo” includes chloro, bromo, and iodo.

R₁ and R₂ can combine with the carbon to which they are attached to complete a carbocyclic ring such as cyclopropyl, cyclobutyl, cyclopentyl and cyclohexyl. Similarly, R₃ and R₄ can be taken together with the carbon to which they are attached to complete a C₃-C₆ carboxylic ring such as cyclopropyl, cyclohexyl, and the like.

As used herein “proinflammatory cytokine induced CRP production” means CRP levels outside the liver or hepatocytes are increased in response to one or more proinflammatory cytokines. “Production” includes all increases in CRP levels regardless of the mechanism by which the level is increased. Mechanisms by which CRP levels are increased include, but are not limited to, secretion of CRP from the liver, increased transcription and/or translation of CRP and stability of CRP protein and/or mRNA. One skilled in the art can determine whether a compound inhibits proinflammatory cytokine induced CRP production by using methods known in the art, for example, as described in Example 16.

As used herein “effective amount” of a compound or pharmaceutical composition means an amount of the compound or pharmaceutical composition that achieves the desired effect for which it is administered. For example, in the context of a method for lowering plasma CRP levels comprising administering to a mammal, in need thereof, an effective amount of a dialkyl ether, or a pharmaceutically acceptable salt thereof, an “effective amount” is an amount of the dialkyl ether that lowers plasma CRP levels in the mammal to which the compound is administered. CRP levels can be measured by methods known in the art. In the context of a method for reducing systemic inflammation comprising administering to a mammal, in need thereof, an effective amount of a compound an “effective amount” is an amount of a compound that reduces systemic inflammation in the mammal to which the compound is administered for example for a compound of formula 1, an “effective amount”, is an amount of a compound of formula 1 that reduces systemic inflammation in the mammal to which the compound is administered. Reduction in systemic inflammation can be measured by comparing the level of a marker of systemic inflammation in the mammal before and after administration of the compound. Markers of systemic inflammation include, but are not limited to CRP, cytokines such as IL-6, and cellular adhesion molecules such as sICAM. In the context of a method for inhibiting proinflammatory cytokine induced CRP production in a mammal comprising administering to the mammal an effective amount of a pharmaceutical composition comprising a compound of formula 1 or a pharmaceutically acceptable salt thereof and a pharmaceutically acceptable diluent, carrier, or excipient, an “effective amount” is an amount of compound of formula 1 or its pharmaceutically acceptable salt that inhibits proinflammatory cytokine induced CRP production in a mammal. Inhibition of proinflammatory cytokine induced CRP production can be measured by methods know in the art, for example the method described in Example 16.

In one embodiment of the invention, the effective amount is between about 150 mg/day and about 1500 mg/day; in another embodiment the effective amount is between about 150 mg/day and about 900 mg/day, in another embodiment the effective amount is between about 300 mg/day and about 900 mg/day; and in another embodiment the effective amount is between about 600 mg/day and about 900 mg/day. One skilled in the art would recognize that the effective amount might depend upon the baseline characteristics of the patient population chosen, as well as the method in which it is used.

As used herein, “Proinflammatory cytokines” include, but are not limited to IL-6 and IL-1β.

Substituted dialkyl ether, substituted aryl-alkyl ether, substituted dialkyl thioether, substituted dialkyl ketone, or substituted-alkyl compounds useful in an invention method include any aspect or embodiment of the therapeutic compounds described in U.S. Pat. Nos. 3,773,946; 3,930,024; 4,287,200; 4,689,344; 4,711,896; 5,648,387; 5,750,569; 5,756,544; 5,783,600; 6,410,802; 6,459,003; and 6,506,799; U.S. patent application Ser. Nos. 09/976,867; 09/976,938; 09/976,898; 09/976,899; and 10/205,939; United States Patent Application Publication Numbers US 2002/0077316; US 2003/0018013; US 2003/0022865; US 2003/0065195; and US 2003/0078239; and PCT International Application Publication numbers WO 96/30328; WO 98/30530; WO 00/59855; WO 01/55078; WO 02/30860; WO 02/30863; WO 02/30882; and WO 02/30884, which are each hereby incorporated herein by reference.

Examples of substituted dialkyl ethers useful in the present invention include those of Formula I

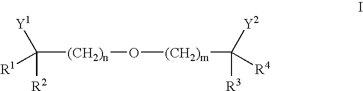

or a pharmaceutically acceptable salt thereof,

where:

n and m independently are integers of from 2 to 9;

R1, R2, R3, and R4 independently are C1-C6 alkyl, C2-C6 alkenyl, or C2-C6 alkynyl; or

R1 and R2 together with the carbon atom to which they are attached, or R3 and R4 together with the carbon atom to which they are attached, or R1 and R2 together with the carbon atom to which they are attached and R3 and R4 together with the carbon atom to which they are attached, can complete a carbocyclic ring having from 3 to 6 carbons;

Y1 and Y2 independently are COOH, CHO, tetrazole, or COOR5, wherein

R5 is C1-C6 alkyl, C2-C6 alkenyl, or C2-C6 alkynyl; and

where the alkyl, alkenyl, and alkynyl groups may be substituted with one or two groups selected from halo, hydroxy, C1-C6 alkoxy, and phenyl;

where halo includes chloro, bromo, and iodo, C1-C6 alkoxy is a C1-C6 alkyl group linked through oxygen.

Additional examples of substituted dialkyl ethers useful in the present invention include those of Formula I where n and m independently are integers of from 2 to 9; R1, R2, R3, and R4 independently are C1-C6 alkyl; and Y1 and Y2 independently are COOH or COOR5, wherein R5 is C1-C6 alkyl.

Other examples of substituted dialkyl ethers useful in the present invention include named 6,6′-oxybis(2,2-dimethylhexanoic acid), represented by the structure drawn below:

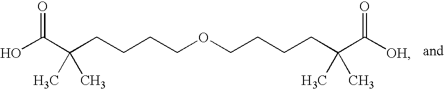

pharmaceutical salts thereof.

An example of a useful salt of named 6,6′-oxybis(2,2-dimethylhexanoic acid) is named 6,6′-oxybis(2,2-dimethylhexanoic acid), calcium salt, represented by the structure drawn below:

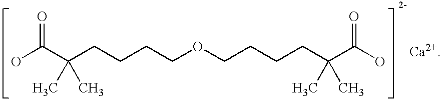

The substituted dialkyl ether named “6,6′-oxybis(2,2-dimethylhexanoic acid), calcium salt” is known by other names, including but not limited to, “6-(5-carboxy-5-methyl-hexyloxy)-2,2-dimethyl-hexanoic acid, monocalcium salt,” “6-(5-carboxy-5-methyl-hexyloxy)-2,2-dimethyl-hexanoic acid, mono-calcium salt, “6-(5-carboxy-5-methyl-hexyloxy)-2,2-dimethyl-hexanoic acid, calcium salt” “CI-1027” and gemcabene calcium. The name “6,6′-oxybis(2,2-dimethylhexanoic acid), calcium salt” is used herein interchangebly with “6-(5-carboxy-5-methyl-hexyloxy)-2,2-dimethyl-hexanoic acid, calcium salt.”

It should be appreciated that the substituted dialkyl ether named 6,6′-oxybis(2,2-dimethylhexanoic acid), calcium salt may exist in a number of different physical forms, including Crystal Form 1 and Crystal Form 2. Crystal Form 1 and Crystal Form 2 of the substituted dialkyl ether named 6-(5-carboxy-5-methylhexyloxy)-2,2-dimethyl-hexanoic acid, calcium salt have been disclosed in PCT International Patent Application Publication No. WO 01/55078. The use of each of these crystal forms is within the scope of the methods of this invention.

It should be appreciated that the substituted dialkyl ether named 6-(5-carboxy-5-methyl-hexyloxy)-2,2-dimethyl-hexanoic acid, calcium salt, may further exist as a hydrate, known by the name 6-(5-carboxy-5-methyl-hexyloxy)-2,2-dimethyl-hexanoic acid, monocalcium salt hydrate in PCT International Patent Application Publication No. WO 01/55078. The use of this or another hydrate form is within the scope of the methods of this invention.

It should be appreciated that the substituted dialkyl ether named 6,6′-oxybis(2,2-dimethylhexanoic acid), calcium salt, may further exist as a C1-C12 alcohol solvate, including an ethyl alcohol, methanol, 1-propyl alcohol, 2-propyl alcohol, or 1-butyl alcohol solvate, known by the names 6-(5-carboxy-5-methylhexyloxy)-2,2-dimethyl-hexanoic acid, mono-calcium salt ethyl alcohol solvate, 6-(5-carboxy-5-methyl-hexyloxy)-2,2-dimethyl-hexanoic acid, mono-calcium salt methanol solvate, 6-(5-carboxy-5-methyl-hexyloxy)-2,2-dimethyl-hexanoic acid, monocalcium salt 1-propyl alcohol solvate, 6-(5-carboxy-5-methyl-hexyloxy)-2,2-dimethyl-hexanoic acid, monocalcium salt 2-propyl alcohol solvate, 6-(5-carboxy-5-methyl-hexyloxy)-2,2-dimethyl-hexanoic acid, monocalcium salt 1-butyl alcohol solvate, respectively, in PCT International Patent Application Publication No. WO 01/55078. The use of these and other alcohol solvate forms is within the scope of the methods of this invention.

Further examples of dialkyl ethers of Formula I include

7,7′-oxybis(2,2-dimethylheptanoic acid);

5,5′-oxybis(2,2-dimethylpentanoic acid);

4,4′-oxybis(2,2-dimethylbutanoic acid);

8,8′-oxybis(2,2-dimethyloctanoic acid);

Ethyl 2,2-dimethyl-5-(4-methyl-4-ethoxycarbonylpentyloxy)pentanoate;

Ethyl 2,2-dimethyl-6-(5-methyl-5-ethoxycarbonylhexyloxy)hexanoate,

Methyl 2,2-dimethyl-8-(7-methyl-7-methoxycarbonyloctyloxy)octanoate;

7-(4-methyl-4-hydroxycarbonylpentyloxy)-2,2-dimethylheptanoic acid;

and a pharmaceutically acceptable salts thereof.

Yet further examples of dialkyl ethers of Formula I include

5-(3-Carboxy-3-methyl-butoxy)-2,2-dimethyl-pentanoic acid;

2,2-Diethyl-5-(4-methoxycarbonyl-4-methyl-pentyloxy)-pentanoic acid;

6-(3-Carboxy-3-ethyl-4-methyl-pentyloxy)-2,2-diethyl-hexanoic acid methyl ester;

2-(3-Chloro-propyl)-5-(5-formyl-7-hydroxy-5-methyl-heptyloxy)-2-methyl-pentanoic acid;

6-(5-Carboxy-5-methyl-hexyloxy)-2,2-dimethyl-hexanoic acid;

6-(5-Carboxy-5-ethyl-heptyloxy)-2,2-diethyl-hexanoic acid, bis sodium salt;

6-(5-Butyl-5-methoxycarbonyl-nonyloxy)-2-ethyl-2-methyl-hexanoic acid;

6-(5-Ethoxycarbonyl-6-hydroxy-5-hydroxymethyl-hexyloxy)-2,2-bis-hydroxymethyl-hexanoic acid ethyl ester;

2,2-Dipropyl-6-[5-propyl-5-(1H-tetrazol-5-yl)-octyloxy]-hexanal;

-{4-[4-(1-Carboxycyclopropan-1-yl)-butyloxy]-butyl}-cyclopropanecarboxylic acid;

1-[4-(5,5-Dimethyl-6-oxo-hexyloxy)-butyl]-cyclopentanecarbaldehyde;

2-Benzyl-6-(5,5-dimethyl-6-oxo-hexyloxy)-2-methyl-hexanal;

6-(6-Ethyl-6-formyl-octyloxy)-2,2-dimethyl-hexanoic acid;

7-(5-Carboxy-5-ethyl-6-methyl-heptyloxy)-2-ethyl-2-isobutyl-heptanoic acid;

2-[2-(6-Carboxy-6-hexyl-dodecyloxy)-ethyl]-2-hexyl-octanoic acid;

8-(3-Carboxy-3-isobutyl-5-methyl-hexyloxy)-2,2-dipropyl-octanoic acid, bis potassium salt;

8-(4-Carboxy-4-methyl-pentyloxy)-2,2-diethyl-octanoic acid;

2-Bromomethyl-9-(4-carboxy-4-chloromethyl-5-hydroxy-pentyloxy)-2-iodomethyl-nonanoic acid;

9-(5-Carboxy-5-pentyl-decyloxy)-2,2-bis-methoxymethyl-nonanoic acid, 1:1 salt with triethylamine;

10-(5,5-Dimethyl-6-oxo-hexyloxy)-2,2-dimethyl-decanoic acid;

11-(5-Hexyloxycarbonyl-5-methyl-hexyloxy)-2,2-dimethyl-undecanoic acid ethyl ester;

5-{3-Ethyl-11-[6-Ethyl-6-(1H-tetrazol-5-yl)-octan-1-yloxy]-undecan-3-yl}-tetrazole; and

11-(10-Benzyl-10-carboxy-11-chloro-undecyloxy)-2,2-diethyl-undecanoic acid;

and a pharmaceutically acceptable salts thereof.

Substituted dialkyl ethers of Formula I, and pharmaceutically acceptable salts thereof, including the compound named 6-(5-carboxy-5-methyl-hexyloxy)-2,2-dimethyl-hexanoic acid, calcium salt, are described in U.S. Pat. No. 5,648,387 and its divisionals U.S. Pat. Nos. 5,750,569; 5,756,544; and 5,783,600, and in PCT International Application Publication nos. WO 96/30328; WO 01/55078.

Examples of substituted-alkyl compounds useful in the present invention include those of Formula II

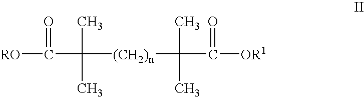

or a pharmaceutically acceptable salt thereof,

where n is 6, 7, 8, 9, or 10; and

R and R1 are selected from the group consisting of hydrogen and C1-C8 alkyl.

Examples of compounds of Formula II include

2,2,9,9-tetramethyldecanedioic acid;

2,2,12,12-tetramethyltridecanedioic acid;

and pharmaceutically acceptable salts thereof.

Substituted-alkyl compounds of Formula II, and pharmaceutically acceptable salts thereof, are described in U.S. Pat. No. 3,773,946.

Examples of substituted-alkyl compounds useful in the present invention include those of Formula III

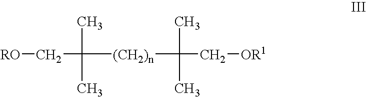

or a pharmaceutically acceptable salt thereof,

where n is 6, 7, 8, 9, or 10;

R and R1 are selected from the group consisting of hydrogen, (C1-C12 alkyl)-C(═O)—, HO2C(CH2)m—CH2—C(═O)—, phenyl-CH2—C(H)(NH2)—C(═O)—, and (HO)₂—P(═O)—; and

m is an integer of from 1 to 3; wherein alkyl is straight or branched.

Examples of compounds of Formula III include

2,2,9,9-tetramethyl-1,10-decanediol;

and a pharmaceutically acceptable salts thereof.

Substituted-alkyl compounds of Formula III, and pharmaceutically acceptable salts thereof, are described in U.S. Pat. No. 3,930,024.

Examples of substituted-aryl alkyl ether compounds useful in the present invention include those of Formula IV

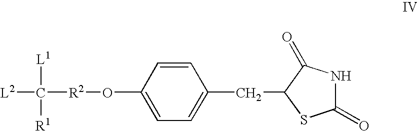

or a pharmaceutically acceptable salt thereof,

where

R1 is C1-C10 alkyl, C3-C7 cycloalkyl, phenyl-(C1-C5 alkyl)-, phenyl, thienyl, furanyl, thiazolyl, pyridinyl, or R3R4N—;

R3 and R4 are the same or different C1-C4 alkyl, or R3 and R4 are combined to each other either directly, or as interrupted by a heteroatom selected from N, O, and S, with the nitrogen atom to which they are both bonded to form a 5- or 6-membered ring, wherein the 5- or 6-membered ring is piperidinyl, morpholinyl, pyrrolidinyl, or piperazinyl;

R2 is a bond or —(CH2)m—;

L1 and L2 are the same or different C1-C4 alkyl, or L1 and L2 are combined to each other to form —(CH2)p—;

p is an integer of from 2 to 6; and

when R1 is C3-C7 cycloalkyl, phenyl-(C1-C5 alkyl)-, phenyl, thienyl, furanyl, thiazolyl, pyridinyl, or R3R4N—, L1 and L2 may further by hydrogen;

where the C3-C7 cycloalkyl, phenyl-(C1-C5 alkyl)-, phenyl, thienyl, furanyl, thiazolyl, pyridinyl, piperidinyl, morpholinyl, pyrrolidinyl, and piperazinyl groups may optionally have from 1 to 3 substituents independently selected from C1-C4 alkyl, (C1-C4 alkyl)-O—, F, Cl, Br, I, OH, and a methylenedioxy group of formula —O—(CH2)m—O—, where the oxygen atoms of the methylenedioxy group are bonded to contiguous carbon atoms to form a ring of from 5 to 7 members; and

each m independently is an integer of from 1 to 3.

Examples of compounds of Formula IV include 5-[4-(1-methylcyclohexylmethyloxy)benzyl]thiazolidine-2,4-dione; a compound of any one of Examples 1 to 8, 10, and 11 of U.S. Pat. No. 4,287,200; any one of Compound Nos. 1 to 54 of Example 10 of U.S. Pat. No. 4,287,200; and any one of Compound Nos. 1 to 7 of Example 12 of U.S. Pat. No. 4,287,200; and pharmaceutically acceptable salts thereof.

Substituted aryl-alkyl ethers of Formula IV, and pharmaceutically acceptable salts thereof, are described in U.S. Pat. No. 4,287,200.

Examples of substituted dialkyl ether, substituted aryl-alkyl ether, substituted dialkyl thioether, substituted dialkyl ketone, or substituted-alkyl compounds useful in the present invention include those of Formula V

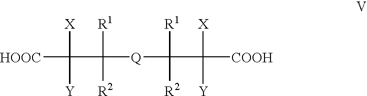

or a pharmaceutically acceptable salt thereof, or an in vivo hydrolyzable functional derivative selected from an ester, amide, or anhydride with (C1-C5 alkyl)-COOH;

where

R1 and R2 each independently represent an unsubstituted or substituted hydrocarbyl selected from C1-C6 alkyl optionally substituted by phenyl, OH, (C1-C6 alkyl)-O—, F, Cl, or Br, C2-C6 alkenyl, C2-C6 alkynyl, C3-C7 cycloalkyl, phenyl optionally substituted by OH, (C1-C6 alkyl)-O—, C1-C6 alkyl, F, Cl, or Br, or heterocyclyl;

X and Y each independently represent hydrogen, C1-C6 alkyl, F, Cl, Br, COOH, (C1-C6 alkyl)-O—C(═O)—, or (C1-C6 alkyl)-N(H)—C(═O)—, and further one of X and Y can also be (C1-C6 alkyl)-O—, HO, or NC—;

Q represents a diradical consisting of an alkylenyl diradical of from 8 to 14 carbon atoms or a heteroalkylenyl diradical of from 8 to 14 members having carbon atoms and a heteroatom selected from S, S(O), S(O)2, N(H), N(C1-C6 alkyl), N(CH2-phenyl), and 0, where the alkylenyl or heteroalkylenyl may optionally be substituted by oxo (═O), F, Cl, Br, OH, or (C1-C6 alkyl)-O—, and where any from 1 to 4 contiguous atoms in the alkylenyl or heteroalkylenyl may comprise a C3-C7 cycloalkyl and where any from 2 to 4 contiguous atoms in the alkylenyl or heteroalkylenyl may comprise a phenyl.

Examples of compounds of Formula V include

2,3,3,14,14,15-hexamethyl-hexadecane-1,16-dioic acid;

2,15-di-carbamoyl-3,3,14,14-tetramethyl-hexadecane-1,16-dioic acid;

3,14-diethyl-3,14-dimethyl-hexadecane-1,16-dioic acid;

3,3,14,14-tetra-(2-propenyl)-hexadecane-1,16-dioic acid;

3,3,14,14-tetra-cyclohexyl-hexadecane-1,16-dioic acid;

2,15-dibromo-3,3,14,14-tetraphenyl-hexadecane-1,16-dioic acid;

1,2-cyclopropylidine-bis-(3,3-dimethyl-7-yl-heptanoic acid):

9,9-pentamethylene-3,3-15,15-tetramethyl-heptadecane-1,17-dioic acid;

1,2-cyclohexylidene-bis-(3,3-dimethyl-7-yl-heptanoic acid);

1,2-phenylene-(3,3-dimethyl-7-yl-heptanoic acid);

3,3,15,15-tetramethyl-9-thia-heptadecane-1,17-dioic acid;

9-oxa-3,3,15,15-tetramethyl-heptadecane-1,17-dioic acid;

9-aza-3,3,15,15-tetramethyl-heptadecane-1,17-dioic acid;

3,3,14,14-tetramethyl-6,11-dithiahexadecane-1,16-dioic acid;

2,15-difluoro-3,3,14,14-tetramethyl-hexadecane-1,16-dioic acid;

2,2,15,15-tetrafluoro-3,3,14,14-tetramethyl-hexadecane-1,16-dioic acid;

2,2,15,15-tetrachloro-3,3,14,14-tetramethyl-hexadecane-1,16-dioic acid;

3,3,14,14-tetrahydroxymethyl-hexadecane-1,16-dioic acid;

2,15-dichloro-3,14-di(chloromethyl)-3,14-dimethyl-hexadecane-1,16-dioic acid;

2,15-dichloro-3,3,14,14-tetra(chloromethyl)-hexadecane-1,16-dioic acid;

3,3,14,14-tetra-(4-hydroxyphenyl)-hexadecane-1,16-dioic acid;

3,3,14,14-tetra-(4-chlorophenyl)-hexadecane-1,16-dioic acid;

3,3,14,14-tetra-(4-methyl-phenyl)-hexadecane-1,16-dioic acid;

3,3,14,14-tetra-(4-methoxy-phenyl)-hexadecane-1,16-dioic acid;

and pharmaceutically acceptable salts thereof.

Additional examples of compounds of Formula V include

1,1,14,14-tetra(ethoxycarbonyl)-2,2,13,13-tetramethyl-tetradecane;

1,1,16,16-tetra(ethoxycarbonyl)-2,2,15,15-tetramethyl-hexadecane;

1,1,12,12-tetra(ethoxycarbonyl)-2,2,11,11-tetramethyl-dodecane;

3,3,14,14-tetramethyl-hexadecane-1,16-dioic acid;

3,3,16,16-tetramethyl-octadecane-1,18-dioic acid;

3,3,12,12-tetramethyl-tetradecane-1,14-dioic acid;

1,14-di-(ethoxycarbonyl)-1,14-dicyano-2,213,13-tetramethyl-tetradecane;

2,15-dicyano-3,3,14,14-tetramethyl-hexadecane-1,16-dioic acid;

2,15-dibromo-3,3,14,14-tetramethyl-hexadecane-1,16-dioic acid;

2,3,3,14,14,15-hexamethyl-hexadecane-1,16-dioic acid;

1,14-diethoxycarbonyl-2,2,13,13-tetramethyl-tetradecane;

1,14-di-(ethoxycarbonyl)-1,14-dibromo-2,2,13,13-tetramethyl-tetradecane;

1,14-bis-carbamoyl-2,2,13,13-tetramethyl-tetradecane;

2,15-dichloro-3,3,14,14-tetramethylhexadecane-1,16-dioic acid;

2,15-dibromo-3,3,14,14-tetramethylhexadecane-1,16-dioic acid;

2,15-dihydroxy-3,3,14,14-tetramethylhexadecane-1,16-dioic acid;

1,14-di-(carbomethoxy)-1,14-dibromo-2,2,13,13-tetramethyltetradecane;

1,14-di-(carbomethoxy)-1,14-dichloro-2,2,13,13-tetramethyltetradecane;

2,15-dimethoxy-3,3,14,14-tetramethylhexadecane-1,16-dioic acid;

1,1,18,18-tetra(carboethoxy)-2,2,17,17-tetramethyloctadecane;

3,3,18,18-tetramethyleicosane-1,20-dioic acid;

3,3,14,14-tetramethyl-8-hexadecene-1,16-dioic acid;

3,3,14,14-tetraphenyl-6,11-diketohexadecane-1,16-dioic acid;

3,3,14,14-tetraphenylhexadecane-1,16-dioic acid;

1,4-phenylene-bis-[(1,1-dimethyl-but-4-yl)-dipropionic acid dimethyl ester];

1,4-phenylene-bis-[(1,1-dimethyl-but-4-yl)-dipropionic acid];

1,4-phenylene-bis(3,3-dimethyl-6-yl-5-hexenoic acid methyl ester);

1,3-phenylene-bis(3,3-dimethyl-6-yl-5-hexenoic acid methyl ester);

1,4-phenylene-bis(3,3-dimethyl-6-yl-hexanoic acid methyl ester);

1,3-phenylene-bis(3,3-dimethyl-6-yl-hexanoic acid methyl ester);

1,4-phenylene-bis(3,3-dimethyl-6-yl-hexanoic acid);

1,3-phenylene-bis(3,3-dimethyl-6-yl-hexanoic acid);

1,4-(cyclohexylidene-bis-(3,3-dimethyl-6-yl-hexanoic acid methyl ester);

1,3-(cyclohexylidene-bis-(3,3-dimethyl-6-yl-hexanoic acid methyl ester);

1,4-(cyclohexylidene-bis-(3,3-dimethyl-6-yl-hexanoic acid);

1,3-(cyclohexylidene-bis-(3,3-dimethyl-6-yl-hexanoic acid);

1,4-phenylene-bis(3,3-dimethyl-7-yl-5-heptenoic acid);

1,3-phenylene-bis(3,3-dimethyl-7-yl-5-heptenoic acid);

1,4-phenylene-bis(3,3-dimethyl-7-yl-heptanoic acid);

1,3-phenylene-bis(3,3-dimethyl-7-yl-heptanoic acid);

1,4-(cyclohexylidene-bis-(3,3-dimethyl-7-yl-heptanoic acid);

1,3-(cyclohexylidene-bis-(3,3-dimethyl-7-yl-heptanoic acid);

1,4-(cyclohexylidene-bis-(3,3-dimethyl-5-oxo-7-yl-heptanoic acid);

and pharmaceutically acceptable salts thereof.

Substituted dialkyl ether, substituted aryl-alkyl ether, substituted dialkyl thioether, substituted dialkyl ketone, or substituted-alkyl compounds of Formula V, and pharmaceutically acceptable salts thereof, are described in U.S. Pat. No. 4,689,344.

Examples of substituted dialkyl ether, substituted aryl-alkyl ether, substituted dialkyl thioether, substituted dialkyl ketone, or substituted-alkyl compounds useful in the present invention include those of Formula VI

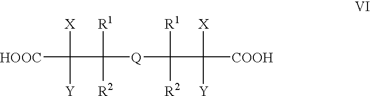

or a pharmaceutically acceptable salt thereof, or an in vivo hydrolyzable functional derivative selected from an ester, amide, or anhydride with (C1-C5 alkyl)-COOH;

where

R1 and R2 each independently represent an unsubstituted or substituted C1-C6 alkyl optionally substituted by OH, (C1-C6 alkyl)-O—, F, Cl, Br, or phenyl, wherein the phenyl optionally substituted one or more times by OH, (C1-C6 alkyl)-O—, C1-C6 alkyl, F, Cl, or Br, C2-C6 alkenyl, C2-C6 alkynyl, C3-C7 cycloalkyl, phenyl optionally substituted by OH, (C1-C6 alkyl)-O—, C1-C6 alkyl, F, Cl, or Br, or heterocycle;

X and Y each independently represent hydrogen, C1-C6 alkyl, (C1-C6 alkyl)-O—, HO, NC—, F, Cl, Br, COOH, (C₁-C₆ alkyl)-O—C(═O)—, or (C₁-C₆ alkyl)-N(H)—C(═O)—;

Q represents a diradical consisting of an alkylenyl diradical of from 8 to 14 carbon atoms or a heteroalkylenyl diradical of from 8 to 14 members having carbon atoms and a heteroatom selected from S.

S(O), S(O)2, N(H), N(C1-C6 alkyl), N(CH2-phenyl), and O, where the alkylenyl or heteroalkylenyl may optionally be substituted by oxo (═O), F, Cl, Br, OH, or (C1-C6 alkyl)-O—, and where any from 1 to 4 contiguous atoms in the alkylenyl or heteroalkylenyl may comprise a C3-C7 cycloalkyl and where any from 2 to 4 contiguous atoms in the alkylenyl or heteroalkylenyl may comprise a phenyl.

Examples of compounds of Formula VI include

2,15-difluoro-3,3,14,14-tetramethyl-1,16-hexadecanedioic acid;

2,15-dichloro-3,3,14,14-tetramethyl-hexadecane-1,16-dioic acid diisopropyl ester;

2,2,15,15-tetrachloro-3,3,14,14-tetramethyl-hexadecane-1,16-dioic acid;

and pharmaceutically acceptable salts thereof.

Substituted dialkyl ether, substituted aryl-alkyl ether, substituted dialkyl thioether, substituted dialkyl ketone, or substituted-alkyl compounds of Formula VI, and pharmaceutically acceptable salts thereof, are described in U.S. Pat. No. 4,711,896.

Examples of substituted dialkyl ether, substituted aryl-alkyl ether, substituted dialkyl thioether, substituted dialkyl ketone, or substituted-alkyl compounds useful in the present invention include those of Formula VII

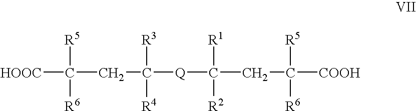

or a pharmaceutically acceptable salt thereof, or in vivo hydrolysable functional derivatives of the carboxylic groups thereof selected from C1-C6 alkyl ester, unsubstituted amide, C1-C6 alkyl amide, bis(C1-C6 alkyl) amide, anhydride with a C1-C6 carboxylic acid, and lactone formed by a dehydrative ring closure between a COOH group and any OH group of R5 or R6,

where

R1, R2, R3, and R4 each independently represents a hydrogen, an unsubstituted or substituted hydrocarbyl radical selected from C1-C6 alkyl, C2-C6 alkenyl, C2-C6 alkynyl, C3-C7 cycloalkyl, phenyl, and phenyl-(C1-C3 alkylenyl), or a heterocyclyl radical;

R5 and R6 independently represent hydrogen, hydroxyl, C1-C6 alkyl, chloro, bromo, cyano, nitro, C1-C6 alkoxy, or CF3;

Q represents a diradical consisting of an unsubstituted or substituted linear chain of 2 to 14 carbon atoms, one or more of which may be replaced by heteroatoms selected from O, S, S(O), S(O)2, N(H), N(C1-C6 alkyl), and N(CH2phenyl);

where substituents are selected from oxo (═O), F, Cl, Br, OH, or (C1-C6 alkyl)-O—, and where any from 1 to 4 contiguous atoms in the in the linear chain may comprise a C3-C7 cycloalkyl and where any from 2 to 4 contiguous atoms in the linear chain may comprise a phenyl.

Additional examples of compounds of Formula VII include those where R1, R2, R3, R4, R5, and R6 are not each hydrogen.

Further examples of compounds of Formula VII include

4,4,11,11-tetramethyltetradecanedioic acid;

diethyl 4,4,13,13-tetramethylhexadeca-2,5,11,14-tetraenedionate;

4,4,13,13-tetramethylhexadecanedioic acid;

4,4,15,15-tetramethyloctadecanedioic acid;

2,2,15,15-tetramethylhexadecanedioic acid;

2,2,17,17-tetramethyloctadecanedioic acid;

and pharmaceutically acceptable salts thereof.

Substituted dialkyl ether, substituted aryl-alkyl ether, substituted dialkyl thioether, substituted dialkyl ketone, or substituted-alkyl compounds of Formula VII, and pharmaceutically acceptable salts thereof, are described in PCT International Patent Application Publication No. WO 98/30530.

Substituted dialkyl ether, substituted aryl-alkyl ether, substituted dialkyl thioether, substituted dialkyl ketone, or substituted-alkyl compounds, and pharmaceutically acceptable salts thereof, are described in U.S. patent application Ser. No. 10/205,939; U.S. Pat. Nos. 6,410,802; 6,459,003; and 6,506,799; in United States Patent Application Publication No. US 2003/0065195; and in PCT International Patent Application Publication No. WO 00/59855.

Substituted dialkyl ether, substituted aryl-alkyl ether, substituted dialkyl thioether, substituted dialkyl ketone, or substituted-alkyl compounds, and pharmaceutically acceptable salts thereof, are described in U.S. patent application Ser. No. 09/976,867; United States Patent Application Publication No. US 2003/0018013; and in PCT International Patent Application Publication No. WO 02/30863.

Substituted dialkyl thioethers are described in U.S. patent application Ser. Nos. 09/976,898; and 09/976,899; United States Patent Application Publication Nos. US 2002/0077316; and US 2003/0022865; and in PCT International Patent Application Publication Nos. WO 02/30882 and WO 02/30884.

Substituted dialkyl ketones are described in U.S. patent application Ser. No. 09/976,938; United States Patent Application Publication No. US 2003/0078239 and PCT International Patent Application Publication No. WO 02/30860.

It should be appreciated that the compounds utilized in an invention method can generally be prepared by carrying out the procedures disclosed in those references above, herein incorporated by reference.

It should be appreciated that the compounds utilized in an invention method are capable of further forming pharmaceutically acceptable salts, including, but not limited to, acid addition and/or base salts. The acid addition salts are formed from basic compounds, whereas the base addition salts are formed from acidic compounds. All of these forms are within the scope of the compounds useful in an invention method, composition, or combination.

Pharmaceutically acceptable acid addition salts of a substituted dialkyl ether, substituted aryl-alkyl ether, substituted dialkyl thioether, substituted dialkyl ketone, or substituted-alkyl compound include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are nontoxic salts of amino acids such as arginate and the like and gluconate, galacturonate (see, for example, Berge S. M. et al., “Pharmaceutical Salts,” J. of Pharma. Sci., 1977;66:1).

An acid addition salt of a substituted dialkyl ether, substituted aryl-alkyl ether, substituted dialkyl thioether, substituted dialkyl ketone, or substituted-alkyl compound is prepared by contacting the free base form of the compound with a sufficient amount of a desired acid to produce a nontoxic salt in the conventional manner. The free base form of the compound may be regenerated by contacting the acid addition salt so formed with a base, and isolating the free base form of the compound in the conventional manner. The free base forms of compounds differ from their respective acid addition salt forms somewhat in certain physical properties such as solubility, crystal structure, hygroscopicity, and the like, but otherwise free base forms of the compounds and their respective acid addition salt forms may be equally utilized in an invention method, composition, or combination.

A pharmaceutically acceptable base addition salt of a substituted dialkyl ether, substituted aryl-alkyl ether, substituted dialkyl thioether, substituted dialkyl ketone, or substituted-alkyl compound may be prepared by contacting the free acid form of the compound with a metal cation such as an alkali or alkaline earth metal cation, or an amine, especially an organic amine. Examples of suitable metal cations include sodium cation (Na+), potassium cation (K+), magnesium cation (Mg2+), calcium cation (Ca2+), and the like. Examples of suitable amines are N,N′-dibenzylethylenediamine, chloroprocaine, choline, diethanolamine, dicyclohexylamine, ethylenediamine, N-methylglucamine, and procaine (see, for example, Berge, supra., 1977).

A base addition salt of a substituted dialkyl ether, substituted aryl-alkyl ether, substituted dialkyl thioether, substituted dialkyl ketone, or substituted-alkyl compound may be prepared by contacting the free acid form of the compound with a sufficient amount of a desired base to produce the salt in the conventional manner. The free acid form of the compound may be regenerated by contacting the salt form so formed with an acid, and isolating the free acid of the compound in the conventional manner. The free acid forms of the compounds differ from their respective salt forms somewhat in certain physical properties such as solubility, crystal structure, hygroscopicity, and the like, but otherwise the salts may be utilized equally in an invention method, composition, or combination.

The compounds useful in an invention method may exist in unsolvated forms as well as solvated forms, including hydrated forms. In general, the solvated forms, including hydrated forms, are equivalent to unsolvated forms. An invention method may utilize any solvated form, including hydrated form, of the compound, as well as mixtures thereof.

The compounds useful in an invention method may possess one or more chiral centers, and each center may exist in the R or S configuration. An invention method may utilize any diastereomeric, enantiomeric, or epimeric form of a substituted dialkyl ether, substituted aryl-alkyl ether, substituted dialkyl thioether, substituted dialkyl ketone, or substituted-alkyl compound, or a pharmaceutically acceptable salt thereof, as well as mixtures thereof.

Certain compounds useful in an invention method may exist as two or more tautomeric forms. Tautomeric forms of the substituted dialkyl ether, substituted aryl-alkyl ether, substituted dialkyl thioether, substituted dialkyl ketone, or substituted-alkyl compounds may interchange, for example, via enolization/de-enolization, 1,2-hydride, 1,3-hydride, or 1,4-hydride shifts, and the like. An invention method may utilize any tautomeric form of the compound, as well as mixtures thereof.

Some compounds useful in an invention method have alkenyl groups, which may exist as entgegen or zusammen conformations, in which case all geometric forms thereof, both entgegen and zusammen, cis and trans, and mixtures thereof, may be utilized in an invention method, composition, or combination.

Some compounds useful in an invention method have cycloalkyl groups, which may be substituted at more than one carbon atom, in which case all geometric forms thereof, both cis and trans, and mixtures thereof, may be used in an invention method, composition, or combination.

Some compounds useful in an invention method may exist as amorphous or crystalline solids, in which case all physical forms thereof, including clathrates thereof and mixtures thereof, may be used in an invention method, composition, or combination.

An invention method may use isotopically-labelled compounds which are identical to those recited above, but for the fact that one or more atoms are replaced by an atom having an atomic mass or mass number different from the atomic mass or mass number usually found in nature. Examples of isotopes that can be incorporated into compounds utilized in an invention method include, but are not limited to, isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively. Certain isotopically labelled compounds, such as with ³H and ¹⁴C, are useful in drug and/or substrate tissue distribution assays. Tritiated, i.e., ³H and carbon-14, i.e., ¹⁴C, isotopes are known for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be utilized in some circumstances. Isotopically labelled compounds of those described above in an invention method can generally be prepared by carrying out the procedures incorporated by reference above and below, or procedures disclosed in the Schemes and/or in the Examples and Preparations, if any, disclosed herein, by substituting a readily available isotopically labelled reagent for a non-isotopically labelled reagent.

Certain of the compounds useful in the present invention can exist in unsolvated forms as well as solvated forms, including hydrated forms. In general, the solvated forms, including hydrated forms, are equivalent to unsolvated forms and are encompassed within the scope of the present invention.

The compounds used in the methods of this invention will be named as alkanoic acids and esters. For example, the compound of the formula

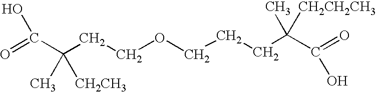

will be named as a pentanoic acid, specifically 2-methyl-2-n-propyl-5-(3-methyl-3-hydroxy-carbonyl)pentoxy pentanoic acid. For compounds wherein n and m in Formula I are the same, and R₁, R₂, R₃, and R₄ are all the same alkyl group and Y₁ and Y₂ both are carboxy groups, the compounds will be named as oxybis alkanoic acids. For example, a compound of the formula

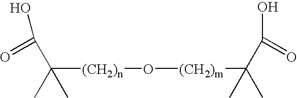

where n and m both are 4, can be named 6,6′-oxybis(2,2-dimethylhexanoic acid).

Typical compounds useful in the methods of the invention are depicted below in Table 1:

TABLE 1

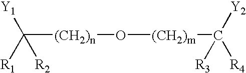

| n | m | R₁ | R₂ | R₃ | R₄ | Y₁ | Y₂ |
|---|---|---|---|---|---|---|---|
| 2 | 3 | CH₃ | CH₃ | CH₃ | CH₃ | COOH | COOH |
| 3 | 3 | CH₃ | CH₃ | Et | Et | COOCH₃ | COOH |
| 2 | 4 | Et | i-Pr | Et | Et | COOH | COOCH₃ |
| 3 | 4 | 3-chloropropyl | CH₃ | CH₃ | 2-hydroxyethyl | COOH | CHO |
| 4 | 4 | CH₃ | CH₃ | CH₃ | CH₃ | COOH | COOH |
| 4 | 4 | Et | Et | Et | Et | COO⁻Na⁺ | COO⁻Na⁺ |
| 4 | 4 | Et | CH₃ | n-Bu | n-Bu | COOH | COOCH₃ |
| 4 | 4 | HOCH₂ | HOCH₂ | HOCH₂ | HOCH₂ | COOEt | COOEt |
| 4 | 4 | n-Pr | n-Pr | n-Pr | n-Pr | tetrazolyl | CHO |
| 4 | 4 | 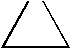 | | 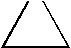 | | COOH | COOH |
| 4 | 4 | 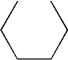 | | CH₃ | CH₃ | CHO | CHO |
| 4 | 4 | phenylmethyl | CH₃ | CH₃ | CH₃ | CHO | CHO |
| 4 | 5 | CH₃ | CH₃ | Et | Et | COOH | CHO |
| 4 | 5 | Et | i-Pr | i-Pr | Et | COOH | COOH |
| 2 | 5 | n-hexyl | n-hexyl | n-hexyl | n-hexyl | COOH | COOH |
| 2 | 6 | i-Bu | i-Bu | n-Pr | n-Pr | COO⁻K⁺ | COO⁻K⁺ |
| 3 | 6 | CH₃ | CH₃ | Et | Et | COOH | COOH |
| 3 | 7 | HOCH₂ | ClCH₂ | BrCH₂ | ICH₂ | COOH | COOH |
| 4 | 7 | n-pentyl | n-pentyl | CH₃OCH₂₋ | CH₃OCH₂₋ | COO⁻Et₃N⁺ | COOH |
| 4 | 8 | CH₃ | CH₃ | CH₃ | CH₃ | CHO | COOH |
| 4 | 9 | CH₃ | CH₃ | CH₃ | CH₃ | COOn-hexyl | COOEt |
| 5 | 8 | Et | Et | Et | Et | tetrazolyl | tetrazolyl |
| 9 | 9 | Et | Et | ClCH₂ | phenylmethyl | COOH | COOH |

The compounds of this invention are prepared utilizing methodology well known in the field of organic chemistry. In a typical synthesis, a carboxy substituted alkyl halide is reacted with a carboxy substituted alkanol in the presence of a base to effect a condensation to provide the invention compound. Carboxy esters typical are utilized, thereby providing invention compounds where Y₁ and Y₂ both are COOR₅. Simple saponification converts one or both of the ester groups to the free acid when desired. The foregoing condensation reaction is depicted as follows:

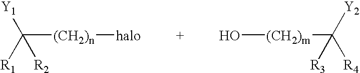

where halo is bromo, chloro, iodo, or the like. The reaction generally is carried out by first reacting the alkanol with about an equimolar quantity of a base such as sodium hydride or metallic sodium, generally in an unreactive organic solvent such as benzene, toluene, xylene, tetrahydrofuran, or the like. This produces the oxide form of the alkanol, which then readily reacts with an equimolar quantity of an alkyl halide to produce an invention compound. The reaction generally is substantially complete within about 2 to about 10 hours when carried out at an elevated temperature of about 50° C. to about 120° C. The invention compound is readily isolated by simply removing the reaction solvent, for instance by evaporation. The product can be purified, if needed, by common methods such as crystallization from solvents such as ethyl acetate, benzene, hexane, and the like, or chromatography, for example, over solid supports such as silica gel.

An alternative method for preparing the invention compounds entails reaction of a di-halo substituted dialkyl ether with an α,α-disubstituted acetic acid or ester, ethanal, or a methyltetrazole. Such reaction is depicted as follows:

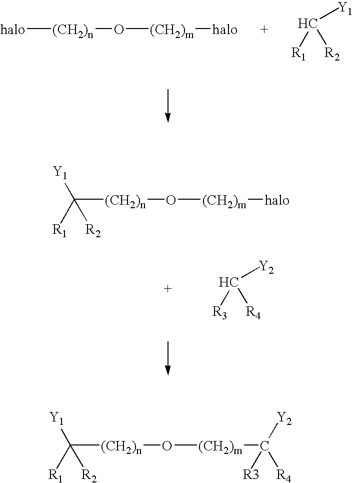

The above process is preferably utilized for preparing invention compounds wherein R₁ and R₂ are the same as R₃ and R₄, respectively, and where Y₁ and Y₂ are the same. In such case, the halo substituted dialkyl ether is reacted with 2 equivalents, or more, of the acetic acid derivative or tetrazole, for example, a compound such as

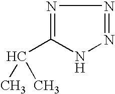

The reaction generally is carried out in a solvent such as tetrahydrofuran, dioxane, diethyl ether, or the like, and in the presence of a base such as sodium hydride, metallic sodium, butyl lithium, or the like. The reaction generally is complete within about 2 to about 10 hours when conducted at a temperature of about 0° C. to about 50° C. The product, a compound of the invention, is readily isolated by removing the reaction solvent, and further purification can be accomplished by routine methods if desired, including chromatography, crystallization, and the like.

It may be desirable, at times, to protect some reactive groups with removable organic radicals so as to prevent unwanted side reactions. For example, hydroxy and free carboxy groups can be derivatized with radicals which eliminate their ability to enter into chemical reactions that are carried out, and wherein the radical can be easily removed when desired to regenerate the free hydroxy or carboxy group. Typical hydroxy and carboxy protecting groups, and methods for their attachment and subsequent removal, are fully discussed by Greene and Wuts in “Protective Groups in Organic Synthesis”, 2nd Ed., John Wiley & Sons, Inc, New York, N.Y., 1991. For example, hydroxy groups are readily protected by conversion to o-benzyl group, which are easily cleaved when desired by hydrogenolysis. Carboxy groups generally are converted to esters, for example, para-nitrobenzyl esters or 2,2,2-trichloroethyl esters. Such ester groups are readily hydrolyzed when desired to afford the free carboxy group.

As noted above, the carboxylic acids of this invention readily form salts by reaction with an inorganic base or organic base. Examples of such salts include, but are not limited to inorganic salts made with bases such as sodium hydroxide, potassium hydroxide, calcium hydroxide, and the like. Typical organic bases include triethylamine, pyridine, methylamine, and the like.

### EXAMPLES

The following detailed examples further illustrate the synthesis and use of compounds of this invention. The examples are illustrative only and are not to be construed as limiting in any respect.

### Example 1

6,6′-Oxybis-(2,2-dimethylhexanoic acid)

To a stirred solution of sodium hydride (28 g of 60% dispersed in mineral oil, 700 mmol) in 600 mL of dry tetrahydrofuran containing 61 g (600 mmol) of diisopropylamine were added to 52.9 g (600 mmol) of isobutyric acid. The reaction mixture was stirred at 24° C. for 30 minutes, and then cooled to 0° C. in an ice/acetone bath. To the cold solution were added 286 mL of a 2.1 M solution of n-butyl lithium (600 mmol), and the mixture was stirred at 0° C. for 1 hour. To the cold stirred reaction mixture were added 59.7 g (297 mmol) of 4,4′-dichlorobutyl ether dropwise over 15 minutes. The mixture was warmed to 24° C. and stirred for 48 hours. The reaction mixture was diluted by addition of 600 mL of water. The aqueous layer was separated, washed with 200 mL of diethyl ether, and then acidified to pH 5.0 (Congo red) with about 150 mL of 6N hydrochloric acid. The aqueous acid solution was extracted three times with 300 mL portions of diethyl ether. The ethereal extracts were combined, washed with brine, dried over MgSO₄, and the solvent was removed by evaporation under reduced pressure to provide the product as an oil. The oil was distilled at 160° C. at 3 mm Hg to provide 66.7 g of 6,6′-oxybis(2,2-dimethylhexanoic acid), mp 49-51° C.

Analysis calculated for C₁₆H₃₀O₅: C, 63.47; H, 9.88. Found: C, 63.75; H, 10.00.

### Examples 2 Through 9

By following the general procedure of Example 1, the following compounds were prepared:

7,7′-oxybis(2,2-dimethylheptanoic acid),

5,5′-oxybis(2,2-dimethylpentanoic acid),

4,4′-oxybis(2,2-dimethylbutanoic acid),

8,8′-oxybis(2,2-dimethyloctanoic acid),

Ethyl 2,2-dimethyl-5-(4-methyl-4-ethoxycarbonylpentyloxy)pentanoate,

Ethyl 2,2-dimethyl-6-(5-methyl-5-ethoxycarbonylhexyloxy)hexanoate,

Methyl 2,2-dimethyl-8-(7-methyl-7-methoxycarbonyloctyloxy)octanoate, and

7-(4-Methyl-4-hydroxycarbonylpentyloxy)-2,2-dimethylheptanoic acid.

Further embodiments of this invention include methods for lowering plasma CRP levels, reducing systemic inflammation and inhibiting proinflammatory cytokine induced CRP production, comprising administering to a mammal, in need thereof, an effective amount of a pharmaceutical composition comprising a compound of Formula 1 or a pharmaceutically acceptable salt thereof and a pharmaceutically acceptable diluent, carrier, or excipient. The compounds can be formulated for convenient oral or parenteral administration. Typical pharmaceutical carriers and excipients utilized in oral formulations include lactose; sucrose; starches such as corn starch and potato starch; cellulose derivatives such as methyl and ethyl cellulose; gelatins; talc; oils such as vegetable oils, sesame oil, cottonseed oil; and glycols such as polyethylene glycol. Oral preparations typically will be in the form of tablets, capsules, emulsions, solutions, and the like. Controlled release formulations, for example, using a polymeric matrix or an osmotic pump, or the like, can also be utilized. Typical formulations will contain from about 5% to about 95% by weight of the active dialkyl ether administered with the excipient or carrier. The pharmaceutical preparation is preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. Flavoring agents such as cherry flavor and orange flavor can be incorporated. The composition can, if desired, also contain other compatible therapeutic agents.

For parenteral administration, the compounds can be formulated with diluents such as isotonic saline, 5% aqueous glucose, and the like, for convenient intramuscular and intravenous delivery. The compounds can also be formulated with waxes and gels in the form of suppositories. The compounds also are well-suited to transdermal delivery, and can be formulated with penetrants and the like in the form of patches. The following example further illustrates typical formulations useful in the methods of this invention.

### Example 10

| Ingredient | Amount |
|---|---|
| 2,2-dimethyl-6-(3-methyl-3- | 1000 g |
| hydroxycarbonylbutyloxy)hexanoic acid, | |
| calcium salt | |
| Lactose | 960 g |
| Magnesium Stearate | 40 g |

The ingredients are blended to uniformity and filled into #4 hard gelatin capsules. Each capsule is filled with 200 mg of the blended mixture and contains 100 mg of active dialkyl ether. The capsules are administered to an adult human at the rate of one to three each day to lower plasma CRP.

### Example 11

| Ingredient | Amount | |
|---|---|---|
| 2,2-dimethyl-6-(6-methyl-6- | 3000 | g |
| ethoxycarbonylheptyloxy)hexanoic acid, | | |
| calcium salt | | |
| Lactose | 750 | g |
| Corn Starch | 300 | g |
| Gelatin | 120 | g |
| Water | 1000 | cc |
| Magnesium Stearate | 20 | g |

The dialkyl ether, lactose, and 150 g of the corn starch are blended with a solution of the gelatin in the water. The wet granulation is screened, dried, and rescreened. The dried granules are blended with the magnesium stearate and the remaining corn starch, and the mixture is compressed into 698 mg tablets using 15/32 inch standard concave punches. Each tablet contains 500 mg of active dialkyl ether.

### Example 12

| Ingredient | Amount | |
|---|---|---|
| 6,6′-oxybis(2,2-dimethylhexanoic acid), calcium salt | 4.0 | g |
| Polyoxyethylene sorbitan monostearate | 0.1 | cc |
| Sodium carboxymethyl cellulose | 0.3 | g |
| Complex Magnesium Aluminum Silicate | 0.5 | g |
| Sugar | 10 | g |
| Glycerin | 2 | cc |
| Sodium benzoate | 0.5 | g |
| Sodium citrate | 0.2 | g |
| Approved red dye | 1 | mg |
| Cherry flavor | 0.02 | cc |
| Distilled water qs | 100 | cc |

The polyoxyethylene sorbitan monostearate can be a product such as polysorbate 60 or Tween 60. The complex magnesium-aluminum silicate is a gel-forming agent. A product such as Veegum H.V. can be used. This substance is hydrated overnight in 10 cc of distilled water. A mixture is prepared from the polyoxyethylene sorbitan monostearate, imitation cherry flavor, 30 cc of distilled water, and the dialkyl ether and passed through a homogenizer. With vigorous stirring, the sugar, glycerin, sodium citrate, sodium benzoate, and sodium carboxymethyl cellulose are added, followed by hydrated complex magnesium-aluminum silicate and a solution of the red dye in 2 cc of water. The resulting suspension is homogenized, adjusted to pH 5.0 with citric acid, and diluted to a final volume of 100 cc with distilled water. A 55-cc oral dosage unit of this suspension contains 200 mg of the active dialkyl ether. If desired, the red dye and imitation cherry flavor can be omitted or replaced by other coloring and flavoring agents.

### Example 13

6,6′-oxbis(2,2-dimethylhexanoic acid) 150 mg Tablet

| | | FORMULA PER |
|---|---|---|
| % w/w | INGREDIENTS | 1000 tablets |
| 71.88 | 6,6′-oxybis(2,2- | 168.92 g |
| | dimethylhexanoic acid), | |
| | calcium salt | |
| 15.32 | Lactose Monohydrate NF | 36.00 g |
| 8.00 | Hydroxypropyl Cellulose | 18.80 g |
| 4.00 | Croscarmellose Sodium | 9.40 g |
| 0.80 | Magnesium Stearate | 1.88 g |
| 100.00 | TO MAKE | 235.00 g |

Film Coating

| % w/w | INGREDIENTS | |
|---|---|---|
| 2.98 | Opadry White YS-1-7040 | 7.00 g |
| 0.02 | Simethicone Emulsion USP | 0.05 g |
| | (30%) | |
| 103.00 | To MAKE | 242.05 g |

An aqueous hydroxypropyl cellulose binder solution was prepared in a low shear mixer. 6,6′-oxybis(2,2-dimethylhexanoic acid) and modified lactose monohydrate were loaded into a fluid bed granulator. A top spray granulation process was performed in the fluid bed granulator by spraying the binder solution. The dried granules were passed through a Comil mill and the screened granules were mixed with croscarmellose sodium in a suitable blender until uniform. Screened magnesium stearate was added into the blender, and mixed until uniform. The final blend was compressed into round shape tables with a suitable tablet press. The tablets were film coated in a suitable coating pan to a weight gain of about 3%.

### Example 14

6,6′-oxybis(2,2-dimethylhexanoic acid) 150 mg Capsules

| | | FORMULA PER |
|---|---|---|
| % w/w | INGREDIENTS | 1000 Capsules |
| 49.67 | 6,6′-oxybis(2,2-dimethylhexanoic acid) | 168.89 g |
| | calcium salt | |
| 26.83 | Lactose Monohydrate NF (Granulac 70) | 91.21 g |
| 20.00 | Cellulose Microcrystalline NF/EP (PH 102) | 68.00 g |
| 3.00 | Croscarmellose Sodium, NF/EP | 10.20 g |
| 0.50 | Magnesium Stearate | 1.70 g |
| — | Size #0 Coni-Snap Capsule | 1.70 g |
| 100.00 | To Make | 340.00 g |

The 6,6′-oxybis(2,2-dimethylhexanoic acid), calcium salt, lactose monohydrate, cellulose microcrystalline, and croscarmellose sodium were screened and the ingredients loaded into a blender and mixed until uniform. The screened magnesium stearate was added and mixed until uniform. 340 mg of the powder blend was encapsulated in Size #0 Coni-Snap capsule shells using a suitable capsule-filling machine.

### Example 15

As noted above, the dialkyl ethers of this invention are useful for lowering plasma CRP levels. The ability of a dialkylether to lower plasma CRP levels was determined using in vivo studies routinely utilized by those skilled in the art.

A randomized, double-blind, placebo-controlled, parallel group, dose-response, multicenter study was conducted at 11 centers in the United States and one center in Canada. Eligible patients with HDL-C level<35 mg/dL (0.9 mmol/L) were selected following a 6-week, single-blind placebo, dietary lead in period conducted according to National Cholesterol Education Program (NCEP) Step 1 Diet. The NCEP Step 1 Diet guidelines are as follows:

| | |
|---|---|
| FAT: | Less than 30% of total calories |
| Saturated Fats: | Less than 10% of total calories |
| Polyunsaturated Fats: | Up to 10% of total calories |
| Monounsaturated Fats: | 10% to 15% of total calories |
| CARBOHYDRATES: | 50 to 60% of total calories |
| PROTEIN: | 10% to 20% of total calories |
| CHOLESTEROL: | Less than 300 mg/day |
| TOTAL CALORIES: | To achieve and maintain |
| | desirable weight |

Eligible patients with HDL-C level<35 mg/dL (0.9 mmol/L) were stratified according to whether mean serum TG level measured at both 2 and 4 weeks prior to randomization was <200 mg/dL (2.3 mmol/L) or ≧200 mg/dL (2.3 mmol/L). Within each TG stratum, patients were randomized to receive either 150, 300, 600, or 900 mg of 6,6′-oxybis(2,2-dimethylhexanoic acid) calcium saltor placebo daily (QD) for 12 weeks.

Patients

Eligible patients were women of non-childbearing potential (naturally postmenopausal or surgically sterilized) or men 18 to 80 years of age with a baseline HDL-C<35 mg/dL. Patients were excluded if they had creatine phosphokinase (CPK)>3 times the upper limit of normal (ULN), a body mass index>35 kg/m², uncontrolled hypertension defined as sitting diastolic blood pressure>95 mm Hg whether taking or not taking an acceptable antihypertensive medication, uncontrolled diabetes mellitus (HbA_{1C>}10%), hepatic dysfunction including asparate aminotransferase (AST) or alanine aminotransferase (ALT)>2 times ULN, renal dysfunction as defined by blood urea nitrogen (BUN) or creatinine>2 times ULN or uncontrolled hypothyroidism (thyroid stimulating hormone>1.5 times ULN). Also excluded were those with a history of gall bladder disease or pancreatitis, a history of consuming>14 alcoholic drinks per week, or those with a known hypersensitivity to lipid-altering drugs. Patients who had myocardial infarction, severe or unstable angina pectoris, coronary artery bypass graft or any other cardiovascular event requiring hospitalization within the last 3 months were also excluded from the study. Patients were not permitted any other lipid-altering drugs during the course of the study and if on prestudy lipid-altering drug therapy, were required to undergo an additional 4-week washout period. Use of isotretinoin, insulin, warfarin, immunosuppressive agents and intermittent systemic steroids were also prohibited.

Sample Analysis

All blood samples were collected following a 12-hour fast and analyzed using methods well known to one of skill in the art; these methods are described briefly as follows. Serum concentrations of cholesterol and triglycerides were measured using an enzymatic, colorimetric assay on a Hitachi 747 analyzer. The HDL-C samples were obtained from the supernatant after precipitation of the non-HDL lipoproteins using heparin and manganese chloride. Concentrations of CRP were measured using immunonephelometry on a nephelometer (Dade Behring, Marburg, Germany). The high sensitivity assay was used to measure CRP (Dade Behring, Marburg, Germany).

Statistical Analyses

A sample size of 15 patients per treatment group was planned to provide >90% power to detect a 30% difference in the percent change in HDL-C from baseline to week 12 between the placebo group and at least one 6,6′-oxybis(2,2-dimethylhexanoic acid), calcium salt dose group in each triglyceridemic stratum. This calculation assumed a Dunnett-adjusted two-sided alpha of 0.05 and a common standard deviation of 18%.

Within each TG stratum, an ANCOVA model with the effects of baseline CRP value, treatment, and site was used to analyze the percent change from baseline at the last visit for CRP by producing least squares (LS) means and p-values. p-values were unadjusted for multiplicity.

The Shapiro-Wilk Test for Normality and visual analysis of the residuals was used to determine if the assumption of normality was reasonable. Since CRP levels in all patients were not normally distributed, median percent changes are presented and Conover's nonparametric ANCOVA was used to analyze the ranked data.

Baseline Demographics

A total of 161 patients were randomized. Of these patients, 67 had TG levels<200 mg/dL (14 randomized to placebo and 53 to active treatment) and 94 had TG levels≧200 mg/dL (18 randomized to placebo and 76 to active treatment). Patient characteristics were generally similar across the TG strata with the obvious exception of the lipid parameters (Table 2).

In patients with TG≧200 or TG<200 mg/dL other baseline mean values were as follows: CRP 2.6 and 2.6 mg/L. The study was completed by 152 patients. Six withdrew due to adverse events and 3 failed to complete the study for administrative or personal reasons. Compliance to study medication regimen was assessed at clinic visits by tablet count and found to be similar among the treatment groups. At the end of the study, 97% of placebo treatment and 96% of the active treatment patients were at least 80% compliant.

Percent change from baseline in CRP levels due to the administration of either 150, 300, 600 or 900 mg of 6,6′-oxybis(2,2-dimethylhexanoic acid), calcium salt or placebo is shown in Table 3. At the 900 mg/day dose, in both the ≧200 mg/dL TG stratum and the <200 mg/dL TG stratum, the levels of CRP were reduced by 57% (p<0.001 vs placebo) and 54% (p<0.05 vs Placebo) respectively. At the 600 mg/day dose in the ≧200 mg/dL TG stratum the CRP level was reduced by 29% (p<0.05 vs Placebo). Although the <200 mg/dL TG stratum at the 600 mg/day dose showed a reduction in the CRP level of 58% this reduction was not statistically significant due to the degree of variation between individual subjects levels.

TABLE 2
Summary of Baseline Characteristics by TG Stratum^{a}

| | TG <200 mg/dL | | | | | TG >200 mg/dL | | | | |
|---|---|---|---|---|---|---|---|---|---|---|
| | | 6,6′-oxybis(2,2-dimethylhexanoic acid). | | | | | 6,6′-oxybis(2,2-dimethylhexanoic acid). | | | |
| | Placebo | 150 mg | 300 mg | 600 mg | 900 mg | Placebo | 150 mg | 300 mg | 600 mg | 900 mg |
| Characteristic | N = 14 | N = 14 | N = 11 | N = 14 | N = 14 | N = 18 | N = 20 | N = 21 | N = 17 | N = 18 |
| Men, % | 100 | 100 | 91 | 86 | 100 | 94 | 90 | 91 | 82 | 94 |
| Age | 50 ± 3 | 55 ± 3 | 64 ± 3 | 51 ± 4 | 50 ± 3.0 | 53 ± 3 | 53 ± 2 | 54 ± 2 | 56 ± 3 | 58 ± 2 |
| Waist | 100 ± 3 | 99 ± 3 | 108 ± 3 | 98 ± 4 | 100 ± 2 | 103 ± 3 | 103 ± 2 | 100 ± 3 | 98 ± 3 | 102 ± 2 |
| Circumference | | | | | | | | | | |
| (cm) | | | | | | | | | | |
| Diabetes, % | 7 | 14 | 18 | 14 | 21 | 28 | 15 | 10 | 24 | 22 |
| HDL-C (mg/dL) | 31 ± 1 | 32 ± 1 | 33 ± 1 | 33 ± 1 | 31 ± 1 | 29 ± 1 | 30 ± 1 | 28 ± 1 | 29 ± 1 | 29 ± 1 |
| LDL-C (mg/dL) | 116 ± 10 | 107 ± 10 | 139 ± 11 | 108 ± 8 | 127 ± 9 | 101 ± 8 | 120 ± 9 | 108 ± 8 | 102 ± 9 | 110 ± 9 |
| TG (mg/dL) | 181 ± 12 | 170 ± 13 | 183 ± 1 | 151 ± 10 | 166 ± 17 | 367 ± 32 | 368 ± 40 | 428 ± 47 | 580 ± 133 | 382 ± 31 |

^{a}Mean ± SE

TABLE 3
6,6′-oxybis(2,2-dimethylhexanoic acid), calcium salt

% change in CRP
levels from base line

| Dose (mg) | TG ≧ 200 mg/dL | TG < 200 mg/dL |
|---|---|---|
| placebo | −10.1 | 0 |
| 150 | −22.2 | −14.3 |
| 300 | −16.2 | −16.0 |
| 600 | −28.6* | −58.3 |
| 900 | −57.1** | −53.8* |

*p < 0.05 versus placebo
**p < 0.001 versus placebo

### EXAMPLE 16

As noted above dialkyl ethers are useful for inhibiting proinflammatory cytokine induced CRP production. Studies have shown that the acute phase response can be recapitulated in cultured human hepatoma cells stimulated with the pro-inflammatory cytokines IL-6 and IL-1 in the presence of the corticosteroid Dexamethasone (Lozanski G, Berthier F, and Kushner I, 1997; Biochem J. 328:271-275). Using a similar hepatoma cell system we evaluated the effect 6,6′-oxybis(2,2-dimethylhexanoic acid) on CRP production by cytokine stimulated human hepatoma PLC/PRF/5 cells.

Materials and Methods

The human hepatoma cell line PLC/PRF/5 (American Type Culture Collection, CRL-8024, Manassas, Va., USA) was maintained in Minimum Essential Medium Eagle modified by ATCC (Cat. No. 30-2003 American Type Culture Collection, Manassas, Va., USA) supplemented with 10% fetal bovine serum (Cat No. 16000-044 Gibco, Grand Island, N.Y. USA). Dexamethasone was purchased from Sigma, St. Louis, Mo., USA, (Cat No. D-8893). IL-6 and IL-1β were purchased from R&D System, Minneapolis, Minn. USA. (Cat. No. 206-L-010, 201-LB005) and CRP Elisa Kit from Alpha Diagnostic International, Inc., San Antonio, Tex. USA. (Cat. No. 1000). The DC protein assay kit was from BioRad Labs, Hercules, Calif. USA (Cat No 500-0116).

Cell Culturing and Drug Treatments

Confluent PLC/PRF/5 monolayers in six-well plates (6 days following splitting) were washed three times with pre-warmed medium. The cells were then treated with 1 ml of medium with or without different doses of 6,6′-oxybis(2,2-dimethylhexanoic acid). After 1 hour the medium were replaced with fresh medium containing cytokines (10 ng/ml IL-6 and 1 ng/ml IL-1), 1 μM dexamethasone and 6,6′-oxybis(2,2-dimethylhexanoic acid) at the doses indicated. After 24 hours of incubation, the media were collected and centrifuged for 5 min at 1000 rpm at room temperature. Supernatants were collected and frozen for CRP and protein analysis. The cells were also used for total cell protein measurements.

CRP Measurements

CRP protein concentrations were determined using a CRP ELISA Kit (Alpha Diagnostic International, Inc. San Antonio, Tex. USA (Cat. No.1000)). CRP reference standard (10 μl) or medium (10 μl) were pipetted into the antibody coated wells, Antibody-enzyme conjugate (100 μl) was then added to each well and the mixtures were incubated at room temp for 60 min. Following this incubation the wells were washed five times with tap water, supplemented with 100 μl of HRP substrate Solution A and 100 μl of HRP substrate Solution B (Solutions A and B were as described by the manufacturer of the CRP kit), and incubated at room temperature for 60 minutes. At the end of this incubation a 50 μl stop solution (as described by the manufacturer of the CRP kit) was added to each well and the plate was used to measure absorbance at 450 nm in a Spectra Max Plus, Molecular Devices spectrophotometer.

Total Cell Protein Measurements

After removal of the media the cells in the six-well plates were used for total cell protein measurements as follows. To the cells and in each well 0.1N NaOH (1 ml) was added and the mixture was frozen at −20 C overnight. Next day the cell lysate was harvested and protein concentrations were determined using a DC Protein assay Kit. Bovine Serum Albumin reference standard (10 μl) or cell lysate (10 μl) were pipetted into the microplate, reagent A (25 μl) and reagent B (200 μl) were then added to each well (reagents A and B were as described by the manufacturer of the DC Protein assay Kit), and incubated at room temperature for 15 minutes. At the end of this incubation the plate was used to measure absorbance at 690 nm in a Spectra Max Plus, Molecular Devices spectrophotometer.

Data Analysis

A CRP standard curve (in ng/ml) was generated by determining 450 nm absorbance values for different amounts of a standard CRP solution, provided by the vendor of the CRP kit, and correcting these values by subtracting the 450 nm absorbance of zero CRP control samples. CRP determinations in experimental samples was done as follows: 10 μl cell media samples in duplicates or triplicates were used to determine 450 nm absorbance. Mean absorbance was then calculated and was corrected by subtracting the 450 nm absorbance obtained from zero CRP controls. These corrected absorbance values were then used to estimate CRP levels in the cell media (in ng/ml) using the CRP standard curve described above. Statistical differences between treatments were determined and evaluated for statistical significance using the Prism statistical program. The validity of the assay was determined by calculating the Z-factor for high throughput screening assays (see Ji-Hu Zhang et al. 1999; J. Biomol Screening, 4:67-73).

Results

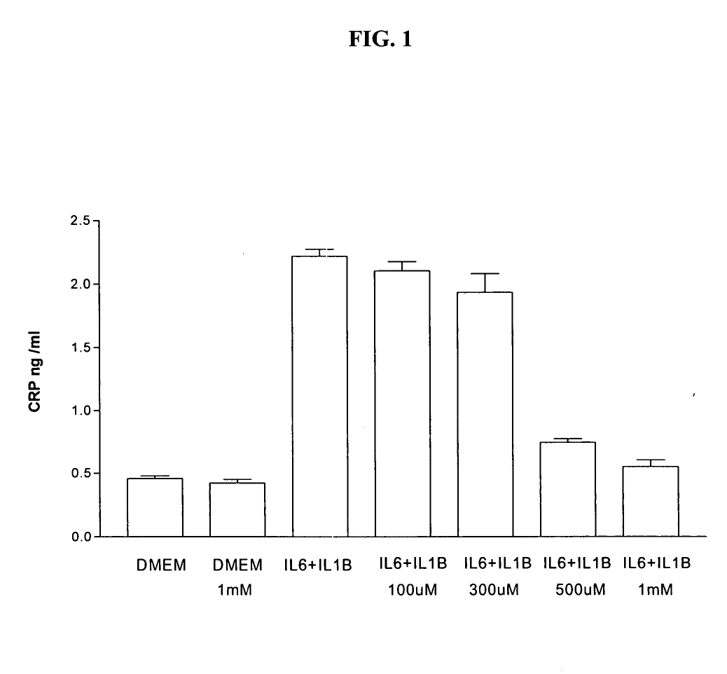

As shown in FIG. 1 (FIG. 1), doses of 500 μM or greater of 6,6′-oxybis(2,2-dimethylhexanoic acid) effectively inhibited proinflammatory cytokine induced CRP production by the human hepatoma cell line PLC/PRF/5 (Z factor=0.47). Not wishing to be bound by theory, these results suggest that 6,6′-oxybis(2,2-dimethylhexanoic acid) interferes with one or more steps in the cytokine signaling pathway responsible for activation of the CRP gene and/or secretion of the protein.

### Example 17

A randomized, double-blind, placebo-controlled, parallel group, dose-response, multicenter study was conducted in hypercholesterolemic patients. The study had three periods: (1) a lipid medication wash-out visit if needed; (2) a qualifying period; and (3) an 8-week double-blind treatment period. The data discussed below is from a portion of a larger study.

Study Population

Men and women who are either:

1) Receiving a statin as monotherapy and who have an LDL-C level>100 mg/dL at the initial clinic wash-out visit; or

2) Receiving no lipid-altering drugs since the initial clinic wash-out visit and who have a mean LDL-C level as follows at 2 qualifying visits:

a. ≧130 mg/dL if NCEP ATP III CHD risk≧10%; or

b. ≧160 mg/dL if NCEP ATP III CHD risk<10%.

Patients with significant cardiac, renal (creatinine>2.0 mg/dL) or liver (alanine aminotransferase [ALT] or aspartate aminotransferase [AST]>1.5× upper limit of normal [ULN]) disease, unexplained CPK levels>3×ULN, body mass index [BMI]>38 kg/m², triglycerides [TG]>400 mg/dL, or age>70 years are excluded as are women who are of childbearing potential, pregnant or lactating. A subset of eligible patients was randomized to receive placebo, 300, 600 or 900 mg/day of 6,6′-oxybis(2,2-dimethylhexanoic acid) calcium salt. Each of these groups had 13 to 15 patients.

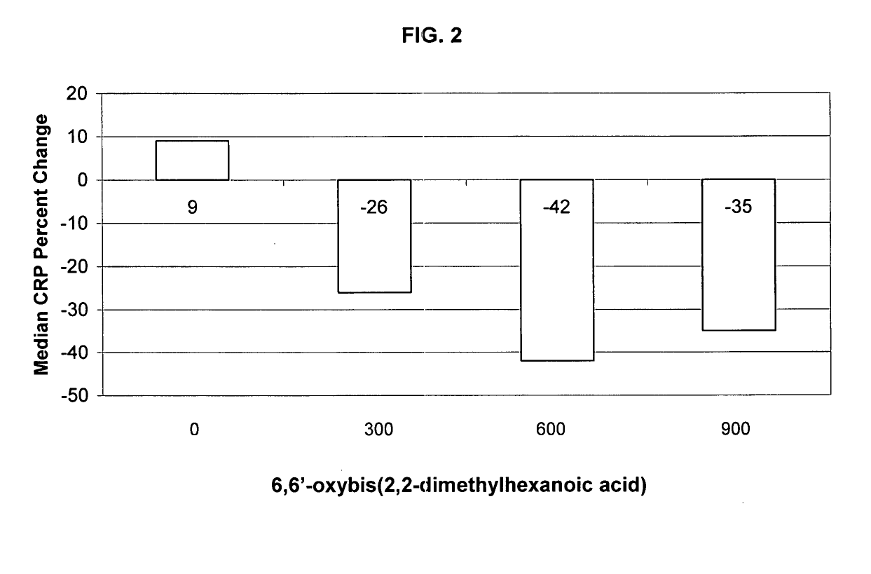

Blood samples were collected at week −2, week −1, randomization and every two weeks thereafter. CRP levels were determined using a high sensitivity assay as in example 15. The mean of the 6 and the 8 week CRP level was calculated for each patient. Values>10 were not included in the analysis as these values are indicative of acute inflammation. For each dosage group the median of the mean values was determined. These median numbers were compared with median CRP values at baseline. Percent change from baseline in CRP levels due to the administration of 300, 600, 900 mg/day of 6,6′-oxybis(2,2-dimethylhexanoic acid) calcium salt or placebo is shown in FIG. 2. At the 300, 600 and 900 mg doses the levels of CRP were reduced by 26% (p=0.16 vs placebo), 42% (p<0.01 vs placebo) and 35% (p<0.01 vs placebo) respectively, compared to a 9.4% increase in the placebo group.

We claim:

1. A method for lowering plasma CRP levels comprising administering to a mammal, in need thereof,
an effective amount of a dialkyl ether, substituted alkyl, substituted aryl-alkyl, substituted dialkyl thioether, substituted dialkyl ketone, substituted-alkyl, or a pharmaceutically acceptable salt of the dialkyl ether, substituted alkyl, substituted aryl-alkyl, substituted dialkyl thioether, substituted dialkyl ketone, or substituted-alkyl:
or a pharmaceutical composition of the dialkyl ether, substituted alkyl, substituted aryl-alkyl, substituted dialkyl thioether, substituted dialkyl ketone, substituted-alkyl, or a pharmaceutically acceptable salt of the dialkyl ether, substituted alkyl, substituted aryl-alkyl, substituted dialkyl thioether, substituted dialkyl ketone, or substituted-alkyl.

2. A method according to claim 1 wherein the mammal is a human.

3. A method according to claim 2 wherein the compound inhibits proinflammatory cytokine induced CRP production.

4. A method for lowering plasma CRP levels comprising administering to a mammal, in need thereof, an effective amount of a compound of the formula: 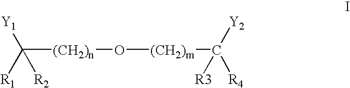
wherein
n and m independently are integers from 2 to 9;
R₁, R₂, R₃, and R₄ independently are C₁-C₆ alkyl, C₂-C₆ alkenyl, C₂-C₆ alkynyl, and R₁ and R₂ together with the carbon to which they are attached, and R₃ and R₄ together with the carbon to which they are attached, independently can complete a carbocyclic ring having from 3 to 6 carbons;
Y₁ and Y₂ independently are COOH, CHO, tetrazole, and COOR₅ where R₅ is C₁-C₆ alkyl, C₂-C₆ alkenyl, C₂-C₆ alkynyl, substituted alkyl, alkenyl, or alkynyl;
and where the alkyl, alkenyl, and alkynyl groups may be substituted with one or two groups selected from halo, hydroxy, C₁-C₆ alkoxy, and phenyl;
or a pharmaceutically acceptable salt thereof.

5. A method according to claim 4 wherein the mammal is a human.

6. A method according to claim 5 wherein the compound inhibits proinflammatory cytokine induced CRP production.

7. A method according to claim 4 wherein the compound is 6,6′-oxybis(2,2-dimethylhexanoic acid) or a pharmaceutically acceptable salt thereof.

8. A method according to claim 7 wherein the mammal is a human.

9. A method for lowering plasma CRP levels comprising administering to a mammal, in need thereof, an effective amount of a pharmaceutical composition comprising a compound of the formula: 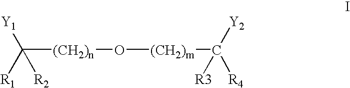
wherein
n and m independently are integers from 2 to 9;
R₁, R₂, R₃, and R₄ independently are C₁-C₆ alkyl, C₂-C₆ alkenyl, C₂-C₆ alkynyl, and R₁ and R₂ together with the carbon to which they are attached, and R₃ and R₄ together with the carbon to which they are attached, independently can complete a carbocyclic ring having from 3 to 6 carbons;
Y₁ and Y₂ independently are COOH, CHO, tetrazole, and COOR₅ where R₅ is C₁-C₆ alkyl, C₂-C₆ alkenyl, C₂-C₆ alkynyl, substituted alkyl, alkenyl, or alkynyl;
and where the alkyl, alkenyl, and alkynyl groups may be substituted with one or two groups selected from halo, hydroxy, C₁-C₆ alkoxy, and phenyl,
or a pharmaceutically acceptable salt thereof;
and a pharmaceutically acceptable diluent, carrier, or excipient.

10. A method according to claim 9 wherein the mammal is a human.

11. A method according to claim 10 wherein the compound inhibits proinflammatory cytokine induced CRP production.

12. A method according to claim 9 wherein the compound is 6,6′-oxybis(2,2-dimethylhexanoic acid) or a pharmaceutically acceptable salt thereof.

13. A method according to claim 12 wherein the mammal is a human.

14. A method for reducing systemic inflammation comprising administering to a mammal, in need thereof,
an effective amount of a dialkyl ether, substituted alkyl, substituted aryl-alkyl, substituted dialkyl thioether, substituted dialkyl ketone, substituted-alkyl, or a pharmaceutically acceptable salt of the dialkyl ether, substituted alkyl, substituted aryl-alkyl, substituted dialkyl thioether, substituted dialkyl ketone, or substituted-alkyl:
or a pharmaceutical composition of the dialkyl ether, substituted alkyl, substituted aryl-alkyl, substituted dialkyl thioether, substituted dialkyl ketone, substituted-alkyl, or a pharmaceutically acceptable salt of the dialkyl ether, substituted alkyl, substituted aryl-alkyl, substituted dialkyl thioether, substituted dialkyl ketone, or substituted-alkyl.

15. A method according to claim 14 wherein the mammal is a human.

16. A method according to claim 15 wherein the compound inhibits proinflammatory cytokine induced CRP production.

17. A method for reducing systemic inflammation comprising administering to a mammal, in need thereof, an effective amount of a compound of the formula: 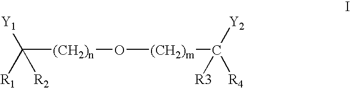
wherein
n and m independently are integers from 2 to 9;
R₁, R₂, R₃, and R₄ independently are C₁-C₆ alkyl, C₂-C₆ alkenyl, C₂-C₆ alkynyl, and R₁ and R₂ together with the carbon to which they are attached, and R₃ and R₄ together with the carbon to which they are attached, independently can complete a carbocyclic ring having from 3 to 6 carbons;
Y₁ and Y₂ independently are COOH, CHO, tetrazole, and COOR₅ where R₅ is C₁-C₆ alkyl, C₂-C₆ alkenyl, C₂-C₆ alkynyl, substituted alkyl, alkenyl, or alkynyl;
and where the alkyl, alkenyl, and alkynyl groups may be substituted with one or two groups selected from halo, hydroxy, C₁-C₆ alkoxy, and phenyl, or a pharmaceutically acceptable salt thereof.

18. A method according to claim 17 wherein the mammal is a human.

19. A method according to claim 18 wherein the compound inhibits proinflammatory cytokine induced CRP production.

20. The method according to claim 17 wherein the compound is 6,6′-oxybis(2,2-dimethylhexanoic acid) or a pharmaceutically acceptable salt thereof.

21. A method according to claim 20 wherein the mammal is a human.

22. A method for reducing systemic inflammation comprising administering to a mammal, in need thereof, an effective amount of a pharmaceutical composition comprising a compound of the formula: 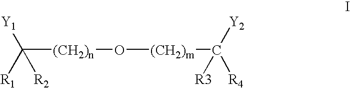
wherein
n and m independently are integers from 2 to 9;
R₁, R₂, R₃, and R₄ independently are C₁-C₆ alkyl, C₂-C₆ alkenyl, C₂-C₆ alkynyl, and R₁ and R₂ together with the carbon to which they are attached, and R₃ and R₄ together with the carbon to which they are attached, independently can complete a carbocyclic ring having from 3 to 6 carbons;
Y₁ and Y₂ independently are COOH, CHO, tetrazole, and COOR₅ where R₅ is C₁-C₆ alkyl, C₂-C₆ alkenyl, C₂-C₆ alkynyl, substituted alkyl, alkenyl, or alkynyl;
and where the alkyl, alkenyl, and alkynyl groups may be substituted with one or two groups selected from halo, hydroxy, C₁-C₆ alkoxy, and phenyl,
or a pharmaceutically acceptable salt thereof;
and a pharmaceutically acceptable diluent, carrier, or excipient.

23. A method according to claim 22 wherein the mammal is a human.

24. A method according to claim 23 wherein the compound inhibits proinflammatory cytokine induced CRP production.

25. A method according to claim 22 wherein the compound is 6,6′-oxybis(2,2-dimethylhexanoic acid) or a pharmaceutically acceptable salt thereof.

26. A method according to claim 25 wherein the mammal is a human.

27. A method of inhibiting proinflammatory cytokine induced CRP production in a mammal, in need thereof, comprising administering to the mammal;
an effective amount of a dialkyl ether, substituted alkyl, substituted aryl-alkyl, substituted dialkyl thioether, substituted dialkyl ketone, substituted-alkyl, or a pharmaceutically acceptable salt of the dialkyl ether, substituted alkyl, substituted aryl-alkyl, substituted dialkyl thioether, substituted dialkyl ketone, or substituted-alkyl:
or a pharmaceutical composition of the dialkyl ether, substituted alkyl, substituted aryl-alkyl, substituted dialkyl thioether, substituted dialkyl ketone, substituted-alkyl, or a pharmaceutically acceptable salt of the dialkyl ether, substituted alkyl, substituted aryl-alkyl, substituted dialkyl thioether, substituted dialkyl ketone, or substituted-alkyl.

28. A method according to claim 27 wherein the mammal is human.

29. A method of inhibiting proinflammatory cytokine induced CRP production in a mammal comprising administering to a mammal an effective amount of a compound of the formula: 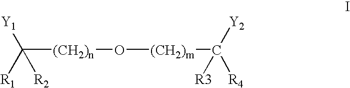
wherein
n and m independently are integers from 2 to 9;
R₁, R₂, R₃, and R₄ independently are C₁-C₆ alkyl, C₂-C₆ alkenyl, C₂-C₆ alkynyl, and R₁ and R₂ together with the carbon to which they are attached, and R₃ and R₄ together with the carbon to which they are attached, independently can complete a carbocyclic ring having from 3 to 6 carbons;
Y₁ and Y₂ independently are COOH, CHO, tetrazole, and COOR₅ where R₅ is C₁-C₆ alkyl, C₂-C₆ alkenyl, C₂-C₆ alkynyl, substituted alkyl, alkenyl, or alkynyl;
and where the alkyl, alkenyl, and alkynyl groups may be substituted with one or two groups selected from halo, hydroxy, C₁-C₆ alkoxy, and phenyl,
or a pharmaceutically acceptable salt thereof.

30. A method according to claim 29 wherein the mammal is a human.

31. A method according to claim 29 wherein the compound is 6,6′-oxybis(2,2-dimethylhexanoic acid) or a pharmaceutically acceptable salt thereof.

32. A method according to claim 31 wherein the mammal is a human.

33. A method of inhibiting proinflammatory cytokine induced CRP production in a mammal comprising administering to a mammal an effective amount of a pharmaceutical composition comprising a compound of the formula: 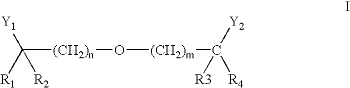
wherein
n and m independently are integers from 2 to 9;
R₁, R₂, R₃, and R₄ independently are C₁-C₆ alkyl, C₂-C₆ alkenyl, C₂-C₆ alkynyl, and R₁ and R₂ together with the carbon to which they are attached, and R₃ and R₄ together with the carbon to which they are attached, independently can complete a carbocyclic ring having from 3 to 6 carbons;
Y₁ and Y₂ independently are COOH, CHO, tetrazole, and COOR₅ where R₅ is C₁-C₆ alkyl, C₂-C₆ alkenyl, C₂-C₆ alkynyl, substituted alkyl, alkenyl, or alkynyl;
and where the alkyl, alkenyl, and alkynyl groups may be substituted with one or two groups selected from halo, hydroxy, C₁-C₆ alkoxy, and phenyl,
or a pharmaceutically acceptable salt thereof;
and a pharmaceutically acceptable diluent, carrier, or excipient.

34. A method of claim 33 wherein the mammal is a human.

35. A method of claim 33 wherein the compound is 6,6′-oxybis(2,2-dimethylhexanoic acid) or a pharmaceutically acceptable salt thereof.

36. A method of claim 35 wherein the mammal is a human.
